(19) United States
(12) Patent Application Publication
(10) Pub. No.: US 2010/0166826 A1
(43) Pub. Date: Jul. 1, 2010

(54) BIODEGRADABLE INTRAVAGINAL DEVICES FOR DELIVERY OF THERAPEUTICS

(75) Inventors: Patrick F. Kiser, Salt Lake City, UT (US); Patrick Tresco, Sandy, UT (US); Mark Mitchnick, East Hampton, NY (US); Kavita Gupta, Salt Lake City, UT (US); Hyder Aliyar, Salt Lake City, UT (US)

Correspondence Address: Ballard Spahr LLP, SUITE 1000, 999 PEACHTREE STREET, ATLANTA, GA, 30309-3915, US

(73) Assignee: University of Uath Research Foundation, Salt Lake City, UT (US)

(21) Appl. No.: 12/523,383

(22) PCT Filed: Jan. 22, 2008
(86) PCT No.: PCT/US08/51694

§ 371 Date: Mar. 8, 2010

Related U.S. Application Data
(60) Provisional application No. 60/881,297, filed on Jan. 19, 2007.

(51) Int. Cl. A61F 2/00; A61F 6/06; A61K 31/675; A61P 31/18; A61M 31/00; A61P 13/02; A61P 15/00; A61P 3/10

(52) U.S. Cl. 424/426; 128/832; 424/430; 514/81; 604/500; 604/285

(57) ABSTRACT

The present disclosure relates to biodegradable intravaginal rings for the delivery of therapeutic or prophylactic agent, inter alia, antiviral agents. The present disclosure further relates to biodegradable polyurethanes which will allow therapeutic/prophylactic agents to be released in a controlled manner that will not degrade when in use, but will degrade upon disposal.

### CROSS REFERENCE TO RELATED APPLICATIONS

The present application is based on and claims priority to U.S. Provisional Application Ser. No. 60/881,297, filed Jan. 19, 2007, the entire contents of which are hereby incorporated by reference.

### FIELD OF THE INVENTION

The present disclosure relates to biodegradable intravaginal devices for the delivery of therapeutic or prophylactic agent, inter alia, antiviral agents. The present disclosure further relates to biodegradable polyurethanes which will allow therapeutic/prophylactic agents to be released in a controlled manner that will not degrade when in use, but will degrade upon disposal.

### BACKGROUND OF THE INVENTION

Current commercially available sustained-delivery intravaginal devices for hormone delivery (See for example, Hussain, A. et al. “The vagina as a route for systemic drug delivery.” Journal of Controlled Release 103, 301-313 (2005)) and those in clinical trials for anti-HIV drug delivery (Malcolm, K. et al. “In vitro release of nonoxynol-9 from silicone matrix intravaginal rings.” J Control Release 91, 355-64 (2003), Malcolm, R. K., Woolfson, A. D., Toner, C. F., Morrow, R. J. & McCullagh, S. D. “Long-term, controlled release of the HIV microbicide TMC120 from silicone elastomer vaginal rings.” J Antimicrob Chemother 56, 954-6 (2005)) consist of non-degradable homopolymers and non-degradable silicone elastomers. These polymers pose disposal problem. The disposal problem may become serious in the future as these devices are also being developed for prophylatic use against sexually transmitted diseases especially prevention of HIV/AIDS worldwide including developing countries. Even if 1% of women worldwide use the current vaginal rings 2-4/year, 180-90 tons/year of non-degradable and non-recyclable waste will be generated. We have invented biodegradable devices for the delivery of therapeutics in the vagina. To this end, we are utilizing thermoplastic polyurethanes (PU) and specifically polyesterurethanes (PEU), for prolonged release of active drug(s), specifically anti-HIV agents. PEU's can be optimized for delivery by varying the input components and their composition to get the desired properties including processing temperature, mechanical properties and degradation kinetics. (See Martin, D. J. et al. The effect of average soft segment length on morphology and properties of a series of polyurethane elastomers. I. Characterization of the series. Journal of Applied Polymer Science 62, 1377-1386 (1996), Martin, D. J. et al. The effect of average soft segment length on morphology and properties of a series of polyurethane elastomers. II. SAXS-DSC annealing study. Journal of Applied Polymer Science 64, 803-817 (1997), Martin, D. J. et al. Effect of soft-segment CH2/O ratio on morphology and properties of a series of polyurethane elastomers. Journal of Applied Polymer Science 60, 557-71 (1996), Martin, D. J. et al. The influence of composition ratio on the morphology of biomedical polyurethanes. Journal of Applied Polymer Science 71, 937-952 (1999), and Szycher, M. Handbook of Polyurethanes (1999)) The device comprises a degradable biocompatible PEU matrix containing a dispersed or dissolved active pharmaceutical ingredient to be released, wherein the PEU may or may not degrade in situ, but degrades ex vivo upon disposal.

### SUMMARY OF THE INVENTION

The present disclosure meets the aforementioned needs in that it has been surprisingly discovered that intravaginal devices can be prepared from certain biodegradable polyurethane co-polymers which can deliver one or more pharmaceutically active compounds, therapeutic agents and/or prophylactic agents and/or contraceptive drugs in a manner that provides slow release over at least about 30-90 days.

The present disclosure in its fullest scope encompasses an intravaginal medical device comprising:

a) a biodegradable polymer; and

b) one or more therapeutic/prophylactic/contraceptive agents;

wherein the medical device is substantially non-biodegradable when in the human body.

The present disclosure provides methods for treating one or more diseases, conditions, symptoms, and the like by providing to a person in need an intravaginal device that can release one or more agents useful and/or effective in treating the one or more diseases, conditions, symptoms, and the like.

### BRIEF DESCRIPTION OF THE FIGURES

FIG. 1 depicts the use cycle of an intravaginal device in the shape of a ring according to the present disclosure.

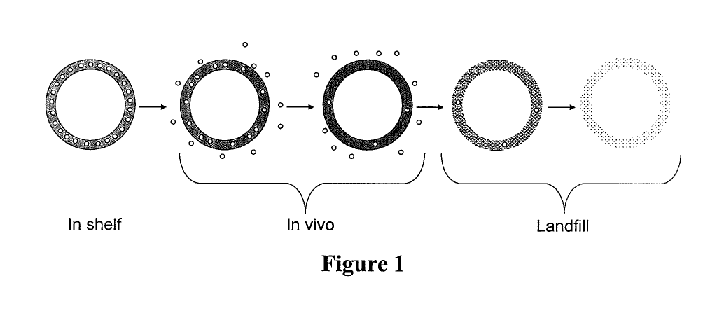

FIG. 2 depicts an embodiment of the present disclosure wherein two separate therapeutic agents are delivered from a single intravaginal ring. FIG. 2 also depicts the manner in which the two extruded portions are affixed to form a ring.

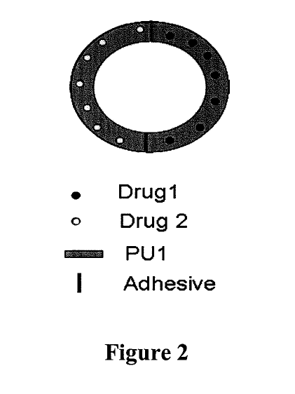

FIG. 3 depicts an embodiment of the present disclosure wherein a slower degrading central core which does not comprise a therapeutic agent is covered with a faster degrading outer layer which comprises one or more therapeutic agents.

FIG. 4 depicts an intravaginal ring comprising a core with one or more therapeutic agents dispersed therein, which is enrobed by a higher melting biodegradable polyurethane (PU) layer.

FIG. 5 depicts the sustained release profile of TMC120, a non-limiting example of an antiviral agent, for a period of one month from a degradable polyurethane made from terathane-co-(caprolactone), 4,4′-methylenebis-cyclohexane diisocyanate and 1,4-butanediol.

FIG. 6 depicts the non-degradability for one month in simulated vaginal conditions of pH 4.2 at 37° C. for a period of one month from a degradable polyurethane made from terathane-co-(caprolactone), 4,4′-methylenebis-cyclohexane diisocyanate and 1,4-butanediol.

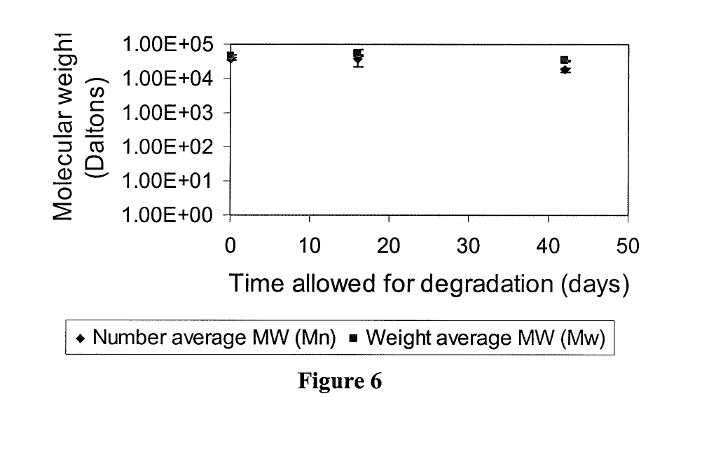

FIG. 7 depicts the mechanical performance of a vaginal ring fabricated from for a period of one month made from terathane-co-(caprolactone), 4,4′-methylenebis-cyclohexane diisocyanate and 1,4-butanediol through force vs. compression-relaxation profile evaluation.

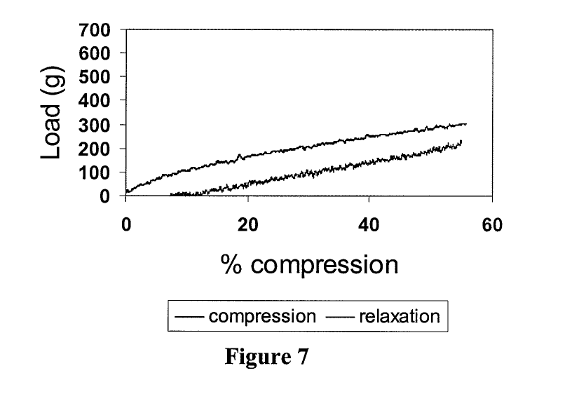

### DETAILED DESCRIPTION OF THE INVENTION

In this specification and in the claims that follow, reference will be made to a number of terms, which shall be defined to have the following meanings:

By “pharmaceutically acceptable” is meant a material that is not biologically or otherwise undesirable, i.e., the material can be administered to an individual along with the relevant active compound without causing clinically unacceptable biological effects or interacting in a deleterious manner with any of the other components of the pharmaceutical composition in which it is contained.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

References in the specification and claims to parts by weight of a particular element or component in a composition or article denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

“Biocompatible” as used herein means the biological response to the material or device is appropriate for the device's intended application in vivo. Any metabolites of these materials should also be biocompatible.

“Biodegradable” is generally referred to herein generally refers to a biocompatible material that will degrade or erode under physiologic conditions to smaller units or chemical species that are, themselves, biocompatible or non-toxic to the subject and capable of being metabolized, eliminated, or excreted by the subject.

“Polymer excipient” as used herein refers to homopolymer or copolymer or blends comprising homopolymers or copolymers and combination thereof that are used as the microparticle wall forming or matrix materials This term should be distinguished from the term “excipient” as defined herein below.

“Polymer” as used herein refers to any type of refers to the biocompatible and/or biodegradable polymers described herein that can be used to fabricate the disclosed vaginal medical devices.

“Absorbable” as used herein means the complete degradation of a material in vivo, and elimination of its metabolites from an animal or human subject.

“Molecular weight” as used herein, unless otherwise specified, refers generally to the relative average molecular weight of the bulk polymer. In practice, molecular weight can be estimated or characterized in various ways including gel permeation chromatography (GPC) or capillary viscometry. GPC molecular weights are reported as the weight-average molecular weight (Mw) or as the number-average molecular weight (Mn). Capillary viscometry provides estimates of molecular weight as the Inherent Viscosity (IV) determined from a dilute polymer solution using a particular set of concentration, temperature, and solvent conditions. Unless otherwise specified, IV measurements are made at 30° C. on solutions prepared in chloroform at a polymer concentration of 0.5 g/dL.

“Controlled release” as used herein means the use of a material to regulate the release of another substance.

“Bioactive agent” is used herein to include a compound of interest contained in or on the microparticle such as therapeutic or biologically active compounds that may be used internally or externally as a medicine for the treatment, diagnosis, cure, or prevention of a disease or disorder. Examples can include, but are not limited to, drugs, small-molecule drugs, peptides, proteins, oligonucleotides. “Bioactive agent” includes a single such agent and is also intended to include a plurality of bioactive agents including, for example, combinations of 2 or more bioactive agents.

“Excipient” is used herein to include any other compound or additive that can be contained in or on the microparticle that is not a therapeutically or biologically active compound. As such, an excipient should be pharmaceutically or biologically acceptable or relevant (for example, an excipient should generally be non-toxic to the subject). “Excipient” includes a single such compound and is also intended to include a plurality of excipients. This term should be distinguished from the term “polymer excipients” as defined above.

“Agent” is used herein to refer generally to compounds that are contained in or on a microparticle composition. Agent can include a bioactive agent or an excipient. “Agent” includes a single such compound and is also intended to include a plurality of such compounds.

The following chemical hierarchy is used throughout the specification to describe and enable the scope of the present disclosure and to particularly point out and distinctly claim the units which comprise the compounds of the present disclosure, however, unless otherwise specifically defined, the terms used herein are the same as those of the artisan of ordinary skill. The term “hydrocarbyl” stands for any carbon atom-based unit (organic molecule), said units optionally containing one or more organic functional group, including inorganic atom comprising salts, inter alia, carboxylate salts, quaternary ammonium salts. Within the broad meaning of the term “hydrocarbyl” are the classes “acyclic hydrocarbyl” and “cyclic hydrocarbyl” which terms are used to divide hydrocarbyl units into cyclic and non-cyclic classes.

As it relates to the following definitions, “cyclic hydrocarbyl” units may comprise only carbon atoms in the ring (carbocyclic and aryl rings) or may comprise one or more heteroatoms in the ring (heterocyclic and heteroaryl). For “carbocyclic” rings the lowest number of carbon atoms in a ring are 3 carbon atoms; cyclopropyl. For “aryl” rings the lowest number of carbon atoms in a ring are 6 carbon atoms; phenyl. For “heterocyclic” rings the lowest number of carbon atoms in a ring is 1 carbon atom; diazirinyl. Ethylene oxide comprises 2 carbon atoms and is a C₂ heterocycle. For “heteroaryl” rings the lowest number of carbon atoms in a ring is 1 carbon atom; 1,2,3,4-tetrazolyl. The following is a non-limiting description of the terms “acyclic hydrocarbyl” and “cyclic hydrocarbyl” as used herein.
A. Substituted and unsubstituted acyclic hydrocarbyl:
   For the purposes of the present disclosure the term “substituted and unsubstituted acyclic hydrocarbyl” encompasses 3 categories of units:
1) linear or branched alkyl, non-limiting examples of which include, methyl (C₁), ethyl (C₂), n-propyl (C₃), iso-propyl (C₃), n-butyl (C₄), sec-butyl (C₄), iso-butyl (C₄), tert-butyl (C₄), and the like; substituted linear or branched alkyl, non-limiting examples of which includes, hydroxymethyl (C₁), chloromethyl (C₁), trifluoromethyl (C₁), aminomethyl (C₁), 1-chloroethyl (C₂), 2-hydroxyethyl (C₂), 1,2-difluoroethyl (C₂), 3-carboxypropyl (C₃), and the like.
2) linear or branched alkenyl, non-limiting examples of which include, ethenyl (C₂), 3-propenyl (C₃), 1-propenyl (also 2-methylethenyl) (C₃), isopropenyl (also 2-methylethen-2-yl) (C₃), buten-4-yl (C₄), and the like; substituted linear or branched alkenyl, non-limiting examples of which include, 2-chloroethenyl (also 2-chlorovinyl) (C₂), 4-hydroxybuten-1-yl (C₄), 7-hydroxy-7-methyloct-4-en-2-yl (C₉), 7-hydroxy-7-methyloct-3,5-dien-2-yl (C₉), and the like.
3) linear or branched alkynyl, non-limiting examples of which include, ethynyl (C₂), prop-2-ynyl (also propargyl) (C₃), propyn-1-yl (C₃), and 2-methyl-hex-4-yn-1-yl (C₇); substituted linear or branched alkynyl, non-limiting examples of which include, 5-hydroxy-5-methylhex-3-ynyl (C₇), 6-hydroxy-6-methylhept-3-yn-2-yl (C₈), 5-hydroxy-5-ethylhept-3-ynyl (C₉), and the like.
B. Substituted and unsubstituted cyclic hydrocarbyl:
   For the purposes of the present disclosure the term “substituted and unsubstituted cyclic hydrocarbyl” encompasses 5 categories of units:
1) The term “carbocyclic” is defined herein as “encompassing rings comprising from 3 to 20 carbon atoms, wherein the atoms which comprise said rings are limited to carbon atoms, and further each ring can be independently substituted with one or more moieties capable of replacing one or more hydrogen atoms.” The following are non-limiting examples of “substituted and unsubstituted carbocyclic rings” which encompass the following categories of units:
   i) carbocyclic rings having a single substituted or unsubstituted hydrocarbon ring, non-limiting examples of which include, cyclopropyl (C₃), 2-methyl-cyclopropyl (C₃), cyclopropenyl (C₃), cyclobutyl (C₄), 2,3-dihydroxycyclobutyl (C₄), cyclobutenyl (C₄), cyclopentyl (C₅), cyclopentenyl (C₅), cyclopentadienyl (C₅), cyclohexyl (C₆), cyclohexenyl (C₆), cycloheptyl (C₇), cyclooctanyl (C₈), decalinyl (C₁₀), 2,5-dimethylcyclopentyl (C₅), 3,5-dichlorocyclohexyl (C₆), 4-hydroxycyclohexyl (C₆), and 3,3,5-trimethylcyclohex-1-yl (C₆).
   ii) carbocyclic rings having two or more substituted or unsubstituted fused hydrocarbon rings, non-limiting examples of which include, octahydropentalenyl (C₈), octahydro-1H-indenyl (C₉), 3a,4,5,6,7,7a-hexahydro-3H-inden-4-yl (C₉), decahydroazulenyl (C₁₀).
   iii) carbocyclic rings which are substituted or unsubstituted bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.
2) The term “aryl” is defined herein as “units encompassing at least one phenyl or naphthyl ring and wherein there are no heteroaryl or heterocyclic rings fused to the phenyl or naphthyl ring and further each ring can be independently substituted with one or more moieties capable of replacing one or more hydrogen atoms.” The following are non-limiting examples of “substituted and unsubstituted aryl rings” which encompass the following categories of units:
   i) C₆ or C₁₀ substituted or unsubstituted aryl rings; phenyl and naphthyl rings whether substituted or unsubstituted, non-limiting examples of which include, phenyl (C₆), naphthylen-1-yl (C₁₀), naphthylen-2-yl (C₁₀), 4-fluorophenyl (C₆), 2-hydroxyphenyl (C₆), 3-methylphenyl (C₆), 2-amino-4-fluorophenyl (C₆), 2-(N,N-diethylamino)phenyl (C₆), 2-cyanophenyl (C₆), 2,6-di-tert-butylphenyl (C₆), 3-methoxyphenyl (C₆), 8-hydroxynaphthylen-2-yl (C₁₀), 4,5-dimethoxynaphthylen-1-yl (C₁₀), and 6-cyano-naphthylen-1-yl (C₁₀).
   ii) C₆ or C₁₀ aryl rings fused with 1 or 2 saturated rings non-limiting examples of which include, bicyclo[4.2.0]octa-1,3,5-trienyl (C₈), and indanyl (C₉).
3) The terms “heterocyclic” and/or “heterocycle” are defined herein as “units comprising one or more rings having from 3 to 20 atoms wherein at least one atom in at least one ring is a heteroatom chosen from nitrogen (N), oxygen (O), or sulfur (S), or mixtures of N, O, and S, and wherein further the ring which comprises the heteroatom is also not an aromatic ring.” The following are non-limiting examples of “substituted and unsubstituted heterocyclic rings” which encompass the following categories of units:
   i) heterocyclic units having a single ring containing one or more heteroatoms, non-limiting examples of which include, diazirinyl (C₁), aziridinyl (C₂), urazolyl (C₂), azetidinyl (C₃), pyrazolidinyl (C₃), imidazolidinyl (C₃), oxazolidinyl (C₃), isoxazolinyl (C₃), isoxazolyl (C₃), thiazolidinyl (C₃), isothiazolyl (C₃), isothiazolinyl (C₃), oxathiazolidinonyl (C₃), oxazolidinonyl (C₃), hydantoinyl (C₃), tetrahydrofuranyl (C₄), pyrrolidinyl (C₄), morpholinyl (C₄), piperazinyl (C₄), piperidinyl (C₄), dihydropyranyl (C₅), tetrahydropyranyl (C₅), piperidin-2-onyl (valerolactam) (C₅), 2,3,4,5-tetrahydro-1H-azepinyl (C₆), 2,3-dihydro-1H-indole (C₈), and 1,2,3,4-tetrahydro-quinoline (C₉).
   ii) heterocyclic units having 2 or more rings one of which is a heterocyclic ring, non-limiting examples of which include hexahydro-1H-pyrrolizinyl (C₇), 3a,4,5,6,7,7a-hexahydro-1H-benzo[d]imidazolyl (C₇), 3a,4,5,6,7,7a-hexahydro-1H-indolyl (C₈), 1,2,3,4-tetrahydroquinolinyl (C₉), and decahydro-1H-cycloocta[b]pyrrolyl (C₁₀)
4) The term “heteroaryl” is defined herein as “encompassing one or more rings comprising from 5 to 20 atoms wherein at least one atom in at least one ring is a heteroatom chosen from nitrogen (N), oxygen (O), or sulfur (S), or mixtures of N, O, and S, and wherein further at least one of the rings which comprises a heteroatom is an aromatic ring.” The following are non-limiting examples of “substituted and unsubstituted heterocyclic rings” which encompass the following categories of units:
   i) heteroaryl rings containing a single ring, non-limiting examples of which include, 1,2,3,4-tetrazolyl (C₁), [1,2,3]triazolyl (C₂), [1,2,4]triazolyl (C₂), triazinyl (C₃), thiazolyl (C₃), 1H-imidazolyl (C₃), oxazolyl (C₃), furanyl (C₄), thiopheneyl (C₄), pyrimidinyl (C₄), 2-phenylpyrimidinyl (C₄), pyridinyl (C₅), 3-methylpyridinyl (C₅), and 4-dimethylaminopyridinyl (C₅)
   ii) heteroaryl rings containing 2 or more fused rings one of which is a heteroaryl ring, non-limiting examples of which include: 7H-purinyl (C₅), 9H-purinyl (C₅), 6-amino-9H-purinyl (C₅), 5H-pyrrolo[3,2-d]pyrimidinyl (C₆), 7H-pyrrolo[2,3-a]pyrimidinyl (C₆), pyrido[2,3-d]pyrimidinyl (C₇), 2-phenylbenzo[d]thiazolyl (C₇), 1H-indolyl (C₈), 4,5,6,7-tetrahydro-1-H-indolyl (C₈), quinoxalinyl (C₈), 5-methylquinoxalinyl (C₈), quinazolinyl (C₈), quinolinyl (C₉), 8-hydroxy-quinolinyl (C₉), and isoquinolinyl (C₉).
5) C₁-C₆ tethered cyclic hydrocarbyl units (whether carbocyclic units, C₆ or C₁₀ aryl units, heterocyclic units, or heteroaryl units) which connected to another moiety, unit, or core of the molecule by way of a C₁-C₆ alkylene unit. Non-limiting examples of tethered cyclic hydrocarbyl units include benzyl C₁-(C₆) having the formula:

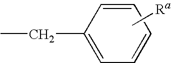

wherein R^{a} is optionally one or more independently chosen substitutions for hydrogen. Further examples include other aryl units, inter alia, (2-hydroxyphenyl)hexyl C₆-(C₆); naphthalen-2-ylmethyl C₁-(C₁₀), 4-fluorobenzyl C₁-(C₆), 2-(3-hydroxy-phenyl)ethyl C₂-(C₆), as well as substituted and unsubstituted C₃-C₁₀ alkylenecarbocyclic units, for example, cyclopropylmethyl C₁-(C₃), cyclopentylethyl C₂-(C₅), cyclohexylmethyl C₁-(C₆);. Included within this category are substituted and unsubstituted C₁-C₁₀ alkylene-heteroaryl units, for example a 2-picolyl C₁-(C₆) unit having the formula:

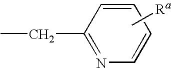

wherein R^{a} is the same as defined above. In addition, C₁-C₁₂ tethered cyclic hydrocarbyl units include C₁-C₁₀ alkyleneheterocyclic units and alkylene-heteroaryl units, non-limiting examples of which include, aziridinylmethyl C₁-(C₂) and oxazol-2-ylmethyl C₁-(C₃).

For the purposes of the present disclosure carbocyclic rings are from C₃ to C₂₀; aryl rings are C₆ or C₁₀; heterocyclic rings are from C₁ to C₉; and heteroaryl rings are from C₁ to C₉.

For the purposes of the present disclosure, and to provide consistency in defining the present disclosure, fused ring units, as well as spirocyclic rings, bicyclic rings and the like, which comprise a single heteroatom will be characterized and referred to herein as being encompassed by the cyclic family corresponding to the heteroatom containing ring, although the artisan may have alternative characterizations. For example, 1,2,3,4-tetrahydroquinoline having the formula:

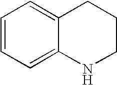

is, for the purposes of the present disclosure, considered a heterocyclic unit. 6,7-Dihydro-5H-cyclopentapyrimidine having the formula:

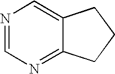

is, for the purposes of the present disclosure, considered a heteroaryl unit. When a fused ring unit contains heteroatoms in both a saturated ring (heterocyclic ring) and an aryl ring (heteroaryl ring), the aryl ring will predominate and determine the type of category to which the ring is assigned herein for the purposes of describing the invention. For example, 1,2,3,4-tetrahydro-[1,8]naphthyridine having the formula:

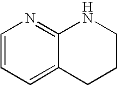

is, for the purposes of the present disclosure, considered a heteroaryl unit.

The term “substituted” is used throughout the specification. The term “substituted” is applied to the units described herein as “substituted unit or moiety is a hydrocarbyl unit or moiety, whether acyclic or cyclic, which has one or more hydrogen atoms replaced by a substituent or several substituents as defined herein below.” The units, when substituting for hydrogen atoms are capable of replacing one hydrogen atom, two hydrogen atoms, or three hydrogen atoms of a hydrocarbyl moiety at a time. In addition, these substituents can replace two hydrogen atoms on two adjacent carbons to form said substituent, new moiety, or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. Three hydrogen replacement includes cyano, and the like. The term substituted is used throughout the present specification to indicate that a hydrocarbyl moiety, inter alia, aromatic ring, alkyl chain; can have one or more of the hydrogen atoms replaced by a substituent. When a moiety is described as “substituted” any number of the hydrogen atoms may be replaced. For example, 4-hydroxyphenyl is a “substituted aromatic carbocyclic ring (aryl ring)”, (N,N-dimethyl-5-amino)octanyl is a “substituted C₈ linear alkyl unit, 3-guanidinopropyl is a “substituted C₃ linear alkyl unit,” and 2-carboxypyridinyl is a “substituted heteroaryl unit.”

The following are non-limiting examples of units which can substitute for hydrogen atoms on a carbocyclic, aryl, heterocyclic, or heteroaryl unit:
i) C₁-C₄ linear or branched alkyl; for example, methyl (C₁), ethyl (C₂), n-propyl (C₃), iso-propyl (C₃), n-butyl (C₄), iso-butyl (C₄), sec-butyl (C₄), and tert-butyl (C₄);
ii) —OR³⁰; for example, —OH, —OCH₃, —OCH₂CH₃, —OCH₂CH₂CH₃;
iii) —C(O)R³⁰; for example, —COCH₃, —COCH₂CH₃, —COCH₂CH₂CH₃;
iv) —C(O)OR³⁰; for example, —CO₂CH₃, —CO₂CH₂CH₃, —CO₂CH₂CH₂CH₃;
v) —C(O)N(R³⁰)₂; for example, —CONH₂, —CONHCH₃, —CON(CH₃)₂;
vi) —N(R³⁰)₂; for example, —NH₂, —NHCH₃, —N(CH₃)₂, —NH(CH₂CH₃);
vii) halogen: —F, —Cl, —Br, and —I;
viii) —CHₘXₙ; wherein X is halogen, m is from 0 to 2, m+n=3; for example, —CH₂F, —CHF₂, —CF₃, —CCl₃, or —CBr₃; and
ix) —SO₂R³⁰; for example, —SO₂H; —SO₂CH₃; —SO₂C₆H₅
   wherein each R³⁰ is independently hydrogen, substituted or unsubstituted C₁-C₄ linear, branched, or cyclic alkyl; or two R³⁰ units can be taken together to form a ring comprising 3-7 atoms. However, substituents which are suitable for replacement of a hydrogen atom are further defined herein below.

### Biodegradable/Biocompatible Polymers

The biodegradable and/or biocompatible polymers disclosed herein the formula:

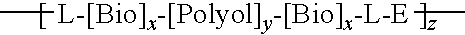

wherein [Bio] is a residue which incorporates into the polyurethane a biologically degradable linkage; [Polyol] is a residue which serves to link [Bio] residues in a biologically degradable manner to form blocks of biologically degradable units; and L is a residue which links the blocks of biologically degradable and/or biologically compatible units together by way of urethane linkages.

Depiction of the polymers according to the present disclosure will have the following format. An example of a polymer according to the present disclosure has the formula:

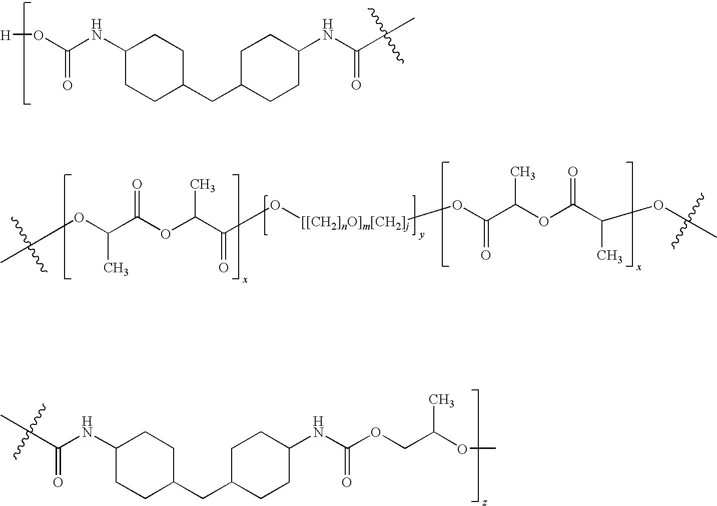

however, when depicting the L linking units, for example the L linking unit in the above formula, the L unit when standing alone will be represented by the generic formula:

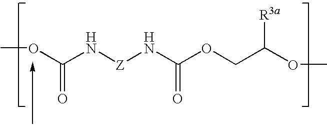

with the understanding that the formulator will know the oxygen atom indicated by the arrow has its origin as part of either a [Bio] unit monomer or a [Polyol] unit monomer.

The first category of [Bio] units which comprise the biodegradable polymers according to the present disclosure are residues the formula:

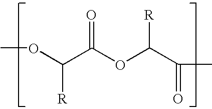

wherein each R is a unit independently chosen from hydrogen, methyl, or ethyl thereby providing units having the formulae:

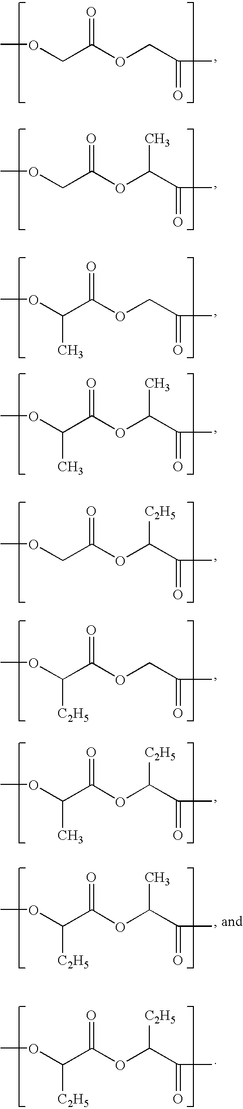

Biodegradable polymers according to the present disclosure which comprise a [Bio] unit having the formula:

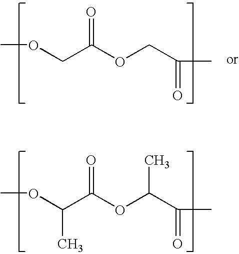

when incorporated into an intravaginal medical device have been shown to be essentially non-biodegradable in the human body, but highly biodegradable when discarded into the environment after use.

The second category of [Bio] units which comprise the biodegradable polymers according to the present disclosure are residues the formula:

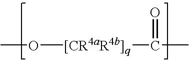

wherein each R^{4a} and R^{4b} is a unit independently chosen from hydrogen, methyl, or ethyl; the index q is from 3 to 7. Non-limiting examples of [Bio] units according to the second category of [Bio] units which comprise the first category of biodegradable polymers include units having the formula:

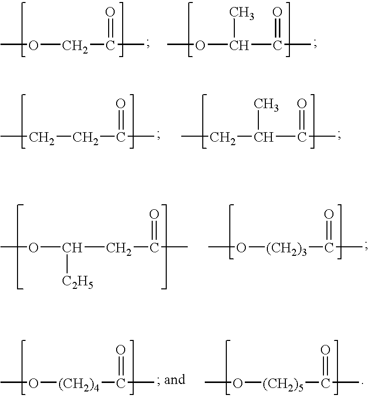

Biodegradable polymers according to the present disclosure which comprise a [Bio] unit having the formula:

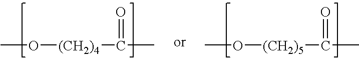

when incorporated into an intravaginal medical device, have been shown to be essentially non-biodegradable in the human body, but highly biodegradable when discarded into the environment after use.

The [Polyol] units which comprise the biodegradable polymers according to the present disclosure are residues the formula:

[symbol]

O—[(CR^{1a}R^{1b})ₙO]ₘ—[CR^{2a}R^{2b}]ⱼ

[symbol]

wherein each R^{1a}, R^{1b}, R^{2a}, and R^{2b} is independently chosen from hydrogen, methyl, or ethyl; the index j is from 2 to 10; the index m is from 0 to 100; the index n is from 2 to 6. The indices m, n and j are selected as to provide starting diols which have average molecular weights from about 500 g/mol to about 5000 g/mol, however each category of [Polyol] has its own specific properties, therefore, a [Polyol] derived from the reaction of a diol starting material having an average molecular weight of 1000 from one category may not impart into the final biodegradable polymer the same properties as a diol starting material from a different category of [Polyol]. For example, polyethylene glycol having an average molecular weight of 1000 g/mol (PEG 1000) would be expected by the formulator to impart different properties of softness, hardness, ease of extrusion, and the like, into a final polymer than polyterathane having an average molecular weight of 1000 g/mol.

The first category of [Polyol] units which comprise the biodegradable polymers relates to [Polyol] units which are polyterathane (polytetramethylene ether glycol) units having the formula:

—[—O(CH₂CH₂CH₂CH₂O)ₘ—(CH₂CH₂CH₂CH₂)]—

wherein the index m is from about 6 to about 70, non-limiting examples of which include:

i) —O[(CH₂CH₂CH₂CH₂O)]₇(CH₂CH₂CH₂CH₂)—;

ii) —O[(CH₂CH₂CH₂CH₂O)]₁₂(CH₂CH₂CH₂CH₂)—;

iii) —O[(CH₂CH₂CH₂CH₂)O]₁₈(CH₂CH₂CH₂CH₂)—;

iv) —O[(CH₂CH₂CH₂CH₂)O]₂₃(CH₂CH₂CH₂CH₂)—;

v) —O[(CH₂CH₂CH₂CH₂)O]₃₀(CH₂CH₂CH₂CH₂)—;

vi) —O[(CH₂CH₂CH₂CH₂)O]₃₂(CH₂CH₂CH₂CH₂)—;

vii) —O[CH₂CH₂CH₂CH₂)O]₄₀(CH₂CH₂CH₂CH₂)—; and
viii) —O[(CH₂CH₂CH₂CH₂)O]₄₃(CH₂CH₂CH₂CH₂)—.

Biodegradable polymers according to the present disclosure which comprise a [Polyol] unit having the formula:

-[—O(CH₂CH₂CH₂CH₂O)ₘ—(CH₂CH₂CH₂CH₂)]—

wherein m is about 12.6 or 26.5 thereby providing a terathane having an average molecular weight of about 1000 g/mol and 2000 g/mol respectively, when incorporated into an intravaginal medical device, said polymers have been shown to be essentially non-biodegradable in the human body, but highly biodegradable when discarded into the environment after use.

A second category of [Polyol] units which comprise the biodegradable polymers relates to units form from the reaction of high molecular weight polyethylene glycols (PEG) having the formula:

-[—O[CH₂CH₂O]ₘ(CH₂CH₂)—]-

with [Bio] unit precursors, wherein the index m represents an approximate whole value average number of ethyleneoxy units present such that the starting diols have average an average molecular weight from about 500 g/mol to about 5000 g/mol. For example, PEG 1000 comprises a mixture of polyethylene glycols having an average molecular weight of approximately 1000 g/mol. The value of m which would reflect this approximation is a unit having the formula:

—O[(CH₂CH₂)O]_{21.3}(CH₂CH₂)—

therefore, it is understood by the formulator that a unit described herein below which has an index m equal to 14, also encompasses m values having a range from about 10 about 18, or whichever range of units a supplier of PEG materials describes the range to be. PEG units having an average molecular weight range up to about 4000 are suitable for use in preparing the polymers of the present disclosure.

The following are non-limiting examples of [Polyol] units according to the second category chosen from:

i) —O[(CH₂CH₂)O]₁₀(CH₂CH₂)—;

ii) —O[(CH₂CH₂)O]₂₁(CH₂CH₂)—;
iii) —O[(CH₂CH₂)O]₃₃(CH₂CH₂)—;

iv) —O[(CH₂CH₂)O]₄₄(CH₂CH₂)—;

v) —O[(CH₂CH₂)O]₅₅(CH₂CH₂)—;

vi) —O[(CH₂CH₂)O]₆₇(CH₂CH₂)—;

vii) —O[(CH₂CH₂)O]₇₈(CH₂CH₂)—; and

viii) —O[(CH₂CH₂)O]₁₁₂(CH₂CH₂)—.

A third category of [Polyol] units which comprise the biodegradable polymers of the present disclosure have the formula:

-[—O[CH₂CH₂CH₂O]ₘ(CH₂CH₂CH₂)—]-

non-limiting examples of which are [Polyol] units chosen from:

i) —O[(CH₂CH₂CH₂)O]₇(CH₂CH₂CH₂)—;

ii) —O[(CH₂CH₂CH₂)O]₁₆(CH₂CH₂CH₂)—;

iii) —O[(CH₂CH₂CH₂)O]₂₅(CH₂CH₂CH₂)—;

iv) —O[(CH₂CH₂CH₂)O]₃₃(CH₂CH₂CH₂)—;

v) —O[(CH₂CH₂CH₂)O]₄₂(CH₂CH₂CH₂)—;

vi) —O[(CH₂CH₂CH₂)O]₅₀(CH₂CH₂CH₂)—;

vii) —O[(CH₂CH₂CH₂)O]₅₉(CH₂CH₂CH₂)—; and

viii) —O[(CH₂CH₂CH₂)O]₈₄(CH₂CH₂CH₂)—.

The fourth category of [Polyol] units which comprise the biodegradable polymers relates to [Polyol] units having the formula:

-[—O[(CR^{1a}H)ₙO]ₘ(CR^{2a}H)ⱼ—]-

wherein each R^{1a} and R^{2a} is independently hydrogen or methyl, the indices j, n, and m are selected such that the molecular weight of the mixed alkylene diol starting material is from about 500 g/mol to about 5000 g/mole. Non-limiting examples of this aspect include:

i) —O[(CH₂CH₂)O]₄O[(CH₂CH₂CH₂)O]₅(CH₂CH₂)—;

ii) —O[(CH₂CH₂)O]₅O[(CH₂CH₂CH₂)O]₄(CH₂CH₂CH₂)—;

iii) —O[(CH₂CH₂)O]₆O[(CH₂CH₂CH₂)O]₇(CH₂CH₂)—;

iv) —O[(CH₂CH₂)O]₇O[(CH₂CH₂CH₂)O]₆(CH₂CH₂CH₂)—;

v) —O[(CH₂CH₂)O]₁₀O[(CH(CH₃)CH₂)O]₁₁(CH₂CH₂)—;

vi) —O[(CH₂CH₂)O]₂₀O[(CH(CH₃)CH₂)O]₁₂[CH(CH₃)CH₂]—;

vii) —O[(CH₂CH₂)O]₂₀O[CH(CH₃)CH₂)O]₆(CH₂CH₂)—; and

viii) —O[(CH₂CH₂)O]₂₀O[(CH(CH₃)CH₂)O]₁₈[CH(CH(CH₃)CH₂]—.

The first category of L linking units which comprise the biodegradable polymers according to the present disclosure are residues the formula:

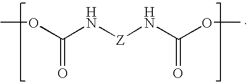

The polymers of the present disclosure comprise two L units per polyurethane unit, one of which is taken together with a chain extender, E unit, to form an -L-E- unit having the formula:

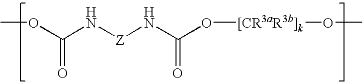

each R^{3a} and R^{3b} is independently chosen from hydrogen, methyl, or ethyl; and the Z units as further defined herein below; and the index k is from 2 to 10.

One aspect of the first category of -L-E- linking units relates to -L-E- units having the formula:

—O—C(O)—NH—Z—NH—C(O)—O—[CR^{3a}H]ₖ—O—

wherein each R^{3a} is independently chosen from hydrogen, methyl, or ethyl and Z can be any Z unit as defined herein below. Non-limiting examples of this first aspect of the first category of -L-E- linking units includes units having the formula:

i) —O—C(O)—NH—Z—NH—C(O)—O—CH₂CH₂—O—;

ii) —O—C(O)—NH—Z—NH—C(O)—O—CH(CH₃)CH₂—O—;

iii) —O—C(O)—NH—Z—H—C(O)—O—CH₂CH(CH₃)—O—;

iv) —O—C(O)—NH—Z—NH—C(O)—O—CH(C₂H₅)CH₂—O—; and

v) —O—C(O)—NH—Z—NH—C(O)—O—CH₂CH(C₂H₅)—O—.

Therefore, when taken together with [Polyol] and [Bio] units which comprise the pre-polymer diols, the polymers will have the general formulae, for example,

—[OC(O)NHZNHC(O)O[Bio]ₓ[Polyol]_{y}[Bio]ₓOC(O)NHZNHC(O)OCH₂CH₂O]—;

—[OC(O)NHZNHC(O)O[Bio]ₓ[Polyol]_{y}[Bio]ₓOC(O)NHZNHC(O)OCH(CH₃)CH₂O]—;

—[OC(O)NHZNHC(O)O[Bio]ₓ[Polyol]_{y}[Bio]ₓOC(O)NHZNHC(O)OCH₂CH(CH₃)O]—;

—[OC(O)NHZNHC(O)O[Bio]ₓ[Polyol]_{y}[Bio]ₓOC(O)NHZNHC(O)OCH(C₂H₅)CH₂O]—;

—[OC(O)NHZNHC(O)O[Bio]ₓ[Polyol]_{y}[Bio]ₓOC(O)NHZNHC(O)OCH₂CH(C₂H₅)O]—;

—[OC(O)NHZNHC(O)O[Bio]ₓ[Polyol]_{y}[Bio]ₓOC(O)NHZNHC(O)O(CH₂)₄O]—.

Z is a unit comprising one or more:

i) substituted or unsubstituted C₁-C₁₂ linear or branched hydrocarbyl:

ii) substituted or unsubstituted C₃-C₇ carbocyclic rings;

iii) substituted or unsubstituted C₁-C₉ heteroaryl rings;

iv) substituted or unsubstituted C₁-C₉ heterocyclic rings; or

v) substituted or unsubstituted C₆, C₁₀, or C₁₋₄ aryl rings;

The first aspect of the first category of L units according to the present disclosure comprises Z units which are:

ii) substituted or unsubstituted C₃-C₇ carbocyclic rings.

The first iteration of this aspect relates to Z units having the formula:

—W—[CR^{5a}R^{5b}]ₜ—Y—

wherein W and Y are each independently an unsubstituted carbocyclic ring chosen from cyclopropyl (C₃), cyclobutyl (C₄), cyclopentyl (C₅), cyclohexyl (C₆), and cycloheptyl (C₇).

Each R^{5a} and R^{5b} is independently chosen from hydrogen, methyl, or ethyl; the index t is from 1 to 3. Non-limiting examples of the first iteration of the first aspect of Z units include units having the formula:

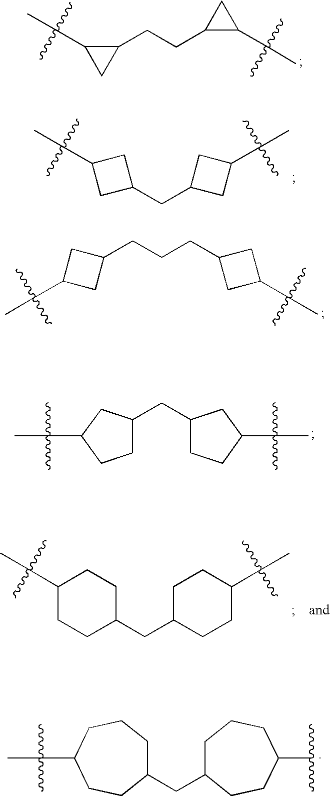

Biodegradable polymers according to the present disclosure which comprise a Z unit having the formula:

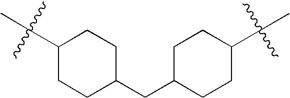

when incorporated into an intravaginal medical device have been shown to be essentially non-biodegradable in the human body, but highly biodegradable when discarded into the environment after use.

The second iteration of this aspect relates to Z units having the formula:

—W—Y—

wherein W and Y are each independently a unsubstituted or unsubstituted carbocyclic ring chosen from cyclopropyl (C₃), cyclobutyl (C₄), cyclopentyl (C₅), cyclohexyl (C₆), and cycloheptyl (C₇). Non-limiting examples of the second iteration of the first aspect of Z units include units having the formula:

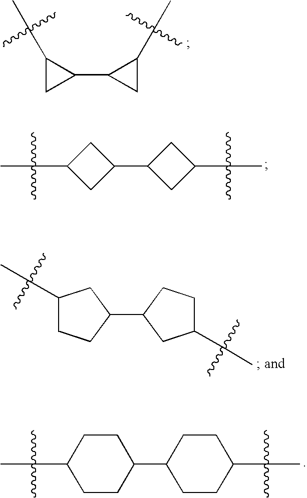

The second aspect of the first category of L units according to the present disclosure comprises Z units which are:

v) substituted or unsubstituted C₆, C₁₀, or C₁₄ aryl rings.

The first iteration of this aspect relates to Z units having the formula:

—W—[CR^{5a}R^{5b}]ₜ—Y—

wherein W and Y are each independently a substituted or unsubstituted carbocyclic ring chosen from phenyl (C₆) or naphthyl (C₁₀) and each R^{5a} and R^{5b} is independently chosen from hydrogen, methyl, or ethyl; the index t is from 1 to 3. Non-limiting examples of the first iteration of this aspect include units having the formula:

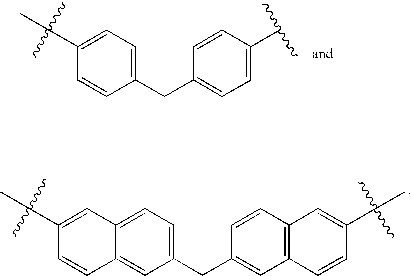

The second iteration of this aspect relates to Z units having the formula:

—W—Y—

wherein W and Y are each independently a substituted or unsubstituted carbocyclic ring chosen from phenyl (C₆) or naphthyl (C₁₀). Non-limiting examples of the second iteration of this aspect include units having the formula:

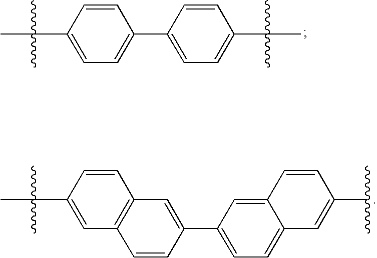

The third aspect of the first category of L units according to the present disclosure comprises Z units which are:

i) substituted or unsubstituted C₁-C₁₂ linear or branched hydrocarbyl.

The first iteration of this aspect relates to Z units having the formula:

—[CH₂]ₜ—

and the index t is from 2 to 12, non-limiting examples of which include units having the formula:

i) —CH₂CH₂—;

ii) —CH₂CH₂CH₂—;

iii) —CH₂CH₂CH₂CH₂—;

iv) —CH₂CH₂CH₂CH₂CH₂—;

v) —CH₂CH₂CH₂CH₂CH₂CH₂—; and

vi) —CH₂CH₂CH₂CH₂CH₂CH₂CH₂—.

The fourth aspect relates to Z units having the formula:

—[CR^{5a}R^{5b}]ₜ—

wherein each R^{5a} and R^{5b} is independently chosen from hydrogen, methyl, ethyl, or —CO₂R⁶, wherein R⁶ is chosen from hydrogen, methyl, or ethyl; the index t is from 2 to 12. Non-limiting examples of this iteration include Z units having the formula:

i) —CH(CH₃)CH₂—;

ii) —CH₂CH(CH₃)—;

iii) —CH(CH₃)CH₂CH₂—;

iv) —CH₂CH(CH₃)CH₂—;

v) —CH₂CH₂CH(CH₃)—;

vi) —CH₂CH₂CH(CH₃)CH₂—;

vii) —CH₂CH(CH₃)CHCH₂—;

viii) —CH₂CH(CH₃)CH(CH₃)CH₂—;

ix) —CH(C₂H₅)CH₂—;

x) —CH₂CH(C₂H₅)—;

xi) —CH(CO₂H)CH₂CH₂—;

xii) —CH₂CH₂CH(CO₂H)—;

xiii) —CH(CO₂H)CH₂CH₂CH₂—;

xiv) —CH₂CH₂CH₂CH(CO₂H)—;

xv) —CH(CO₂H)CH₂CH₂CH₂CH₂—; and

xvi) —CH₂CH₂CH₂CH₂CH(CO₂H)—.

Therefore, the first category of linking units when combined with a chain extender unit, has the formula:

—O—C(O)—NH—W—[CR^{5a}R^{5b}]ₜ—Y—NH—C(O)—O—[CR^{3a}R^{3b}]ₖ—

wherein the index k is from 2 to 12 and the index t is from 1 to 3. The following is a non-limiting example of a -L-E- linking unit comprising the first aspect of Z units:

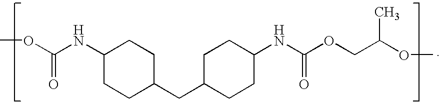

When this linking unit is combined with [Bio] and [Polyol] units according to the present disclosure, the following non-limiting examples of biodegradable polymers suitable for use in the intravaginal medical devices, are formed:

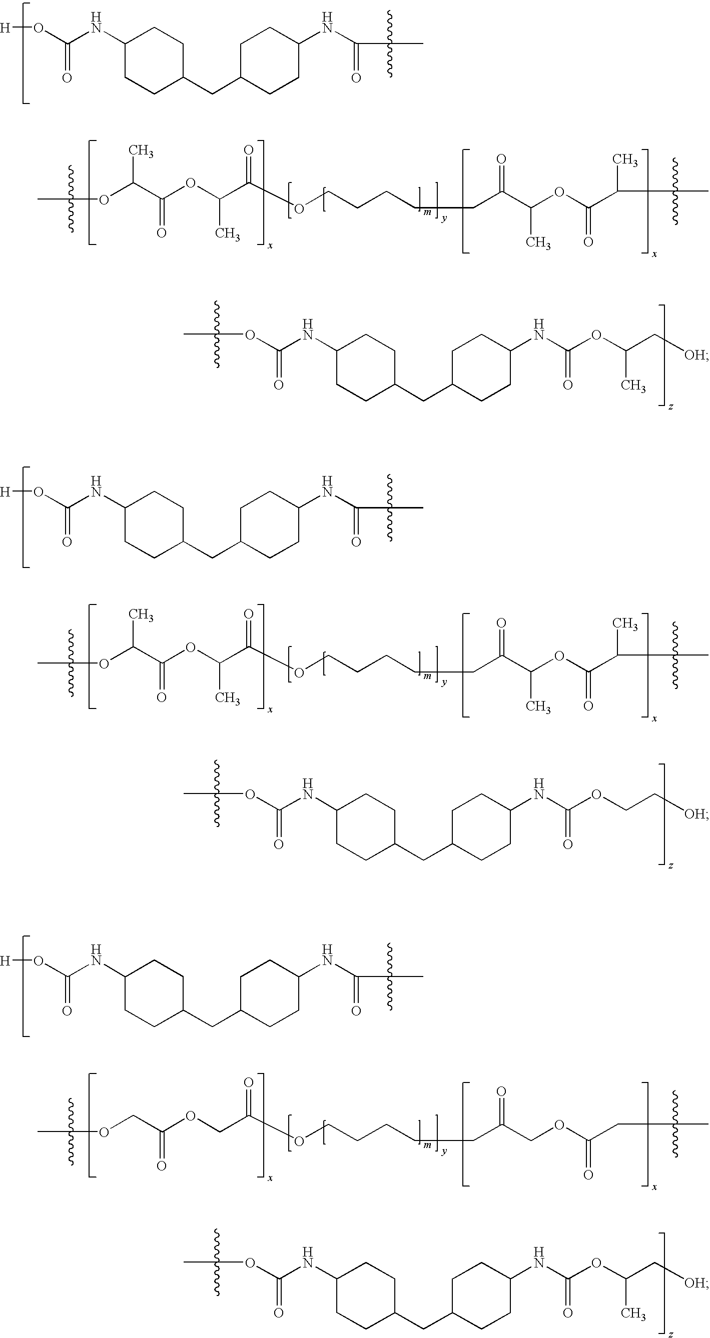

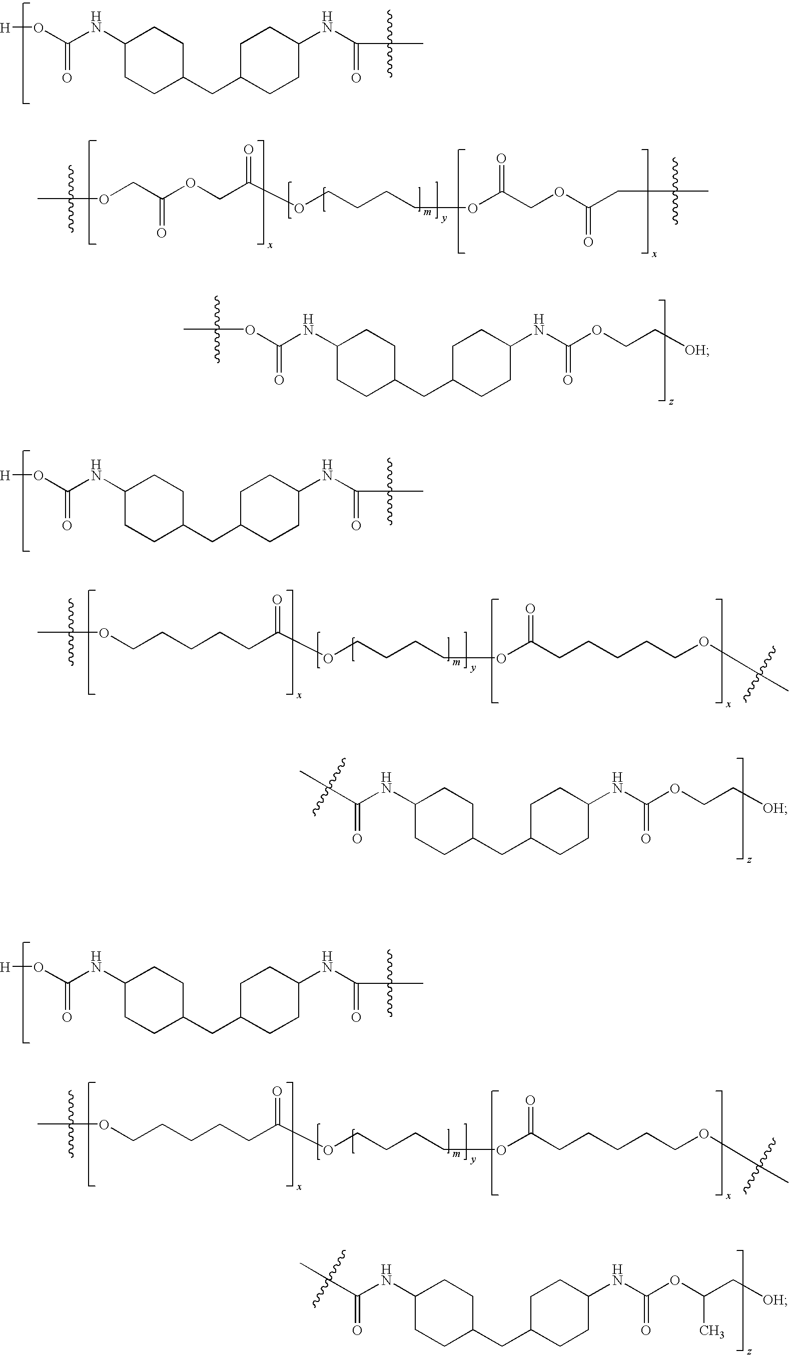

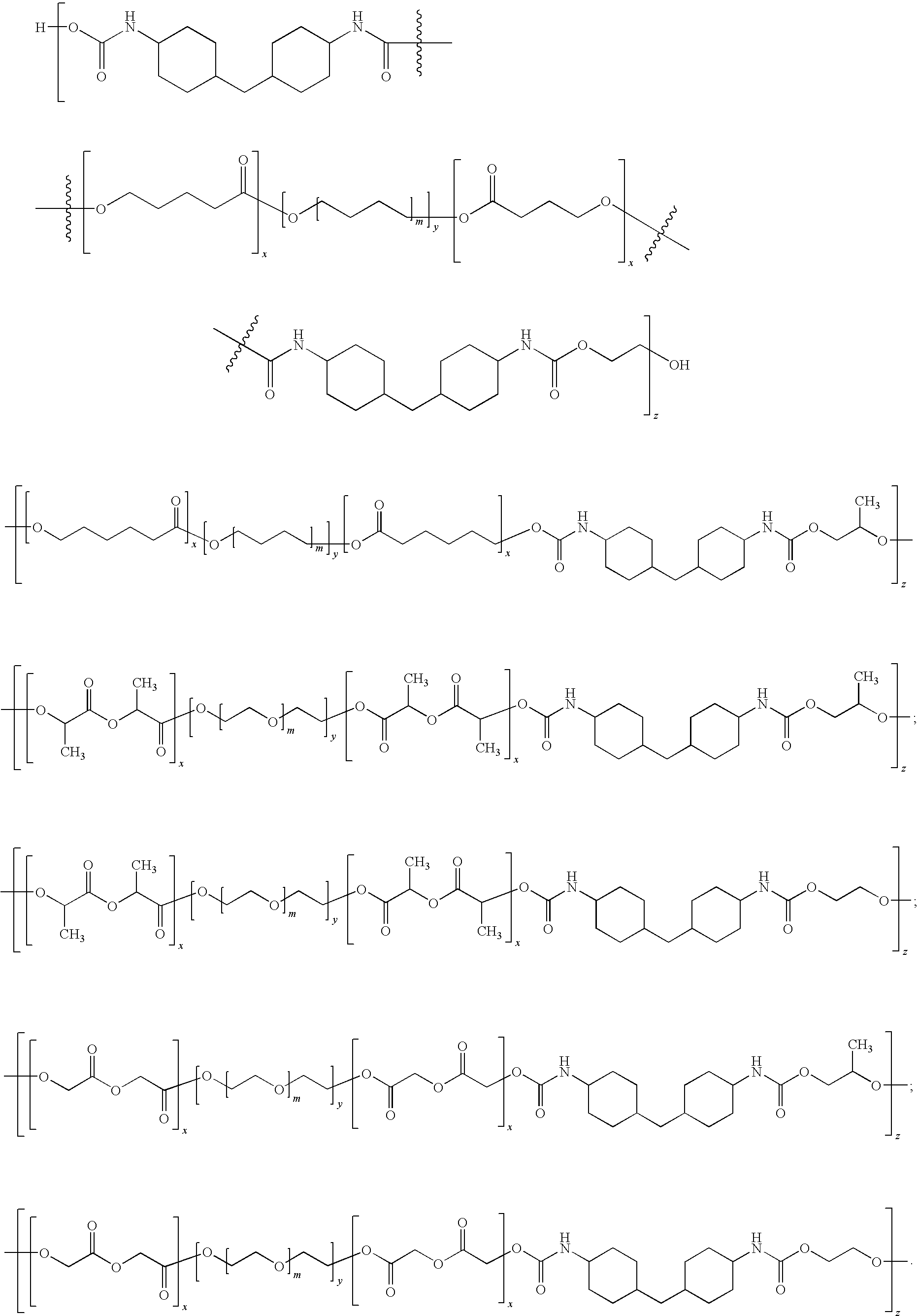

The following are further non-limiting examples of biodegradable polymers according to the present disclosure.

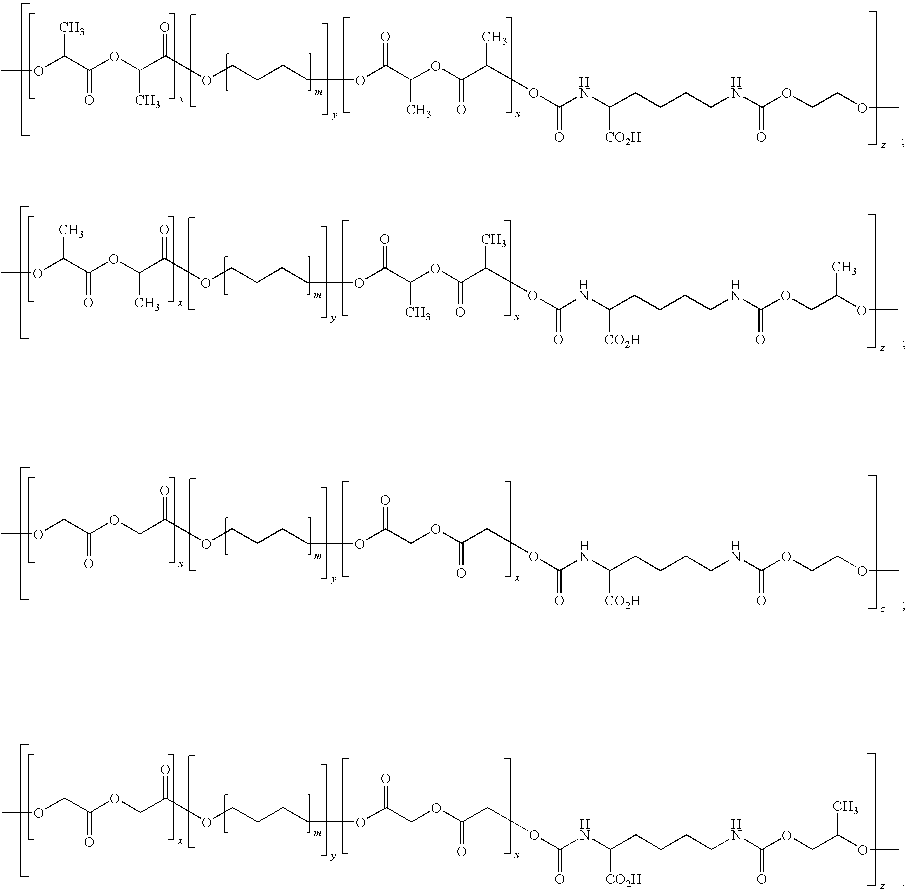

The biodegradable polyurethanes of the present disclosure can be prepared in the following manner.

The following Scheme I depicts a generic procedure for preparing biodegradable polyurethanes according a first embodiment of the present disclosure, wherein the [Bio] units are described herein above and [Polyol] units are also described herein above. In step (a) two equivalents of cyclic ester A are combined with one equivalent of polyol B to form a poly(A)-b-poly(B)-b-poly(A) intermediate C.

### Step (a)

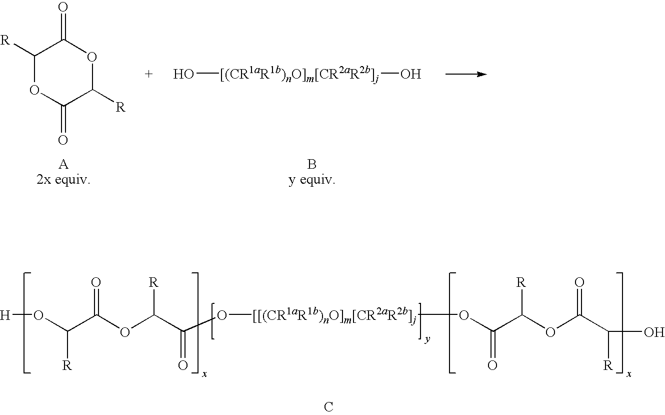

In step (b) intermediate C is reacted with one equivalent of a diisocyanate and one equivalent of a second polyol to form the final biodegradable polyurethane D.

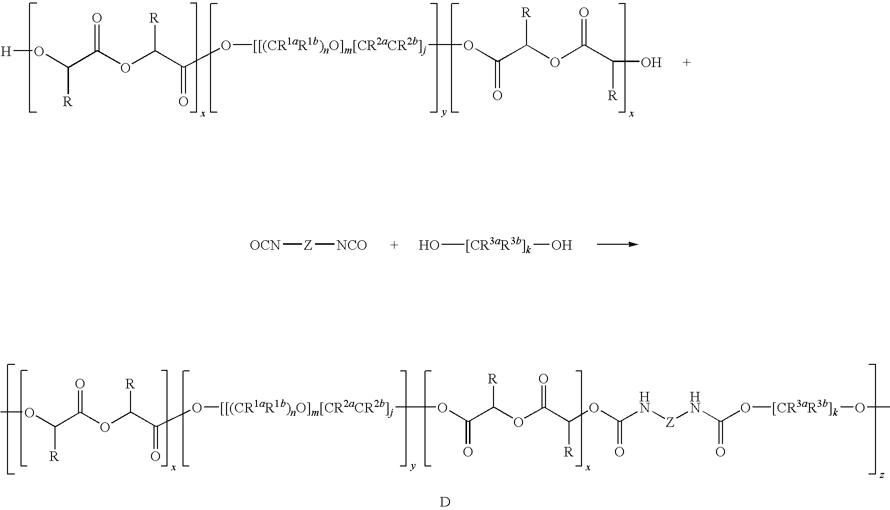

The following Scheme II depicts a generic procedure for preparing biodegradable polyurethanes according a second embodiment of the present disclosure, wherein the [Bio] units are described herein above and [Polyol] units are also described herein above. In step (a) two equivalents of lactone E are combined with one equivalent of polyol B to form a poly(E)-b-poly(B)-b-poly(E) intermediate F.

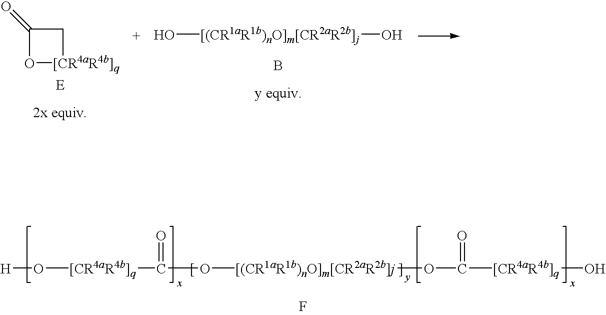

In step (b) intermediate F is reacted with one equivalent of a diisocyanate and one equivalent of a second polyol to form the final biodegradable polyurethane G.

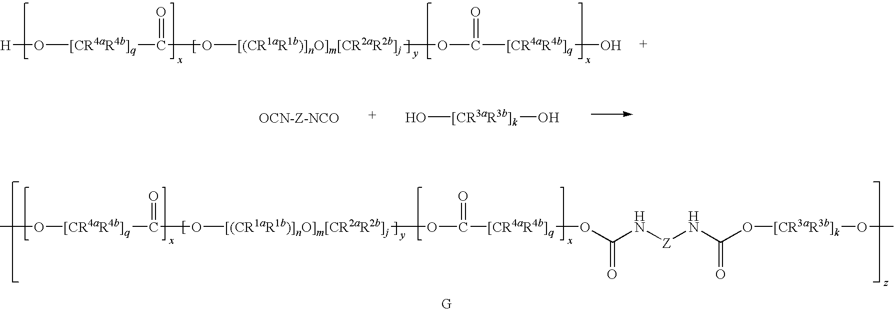

The present disclosure relates to biodegradable polyurethane polymers suitable for use in an intravaginal medical device, said polymers formed by a process comprising:

a) reacting:
i) from 1 to 3 equivalents of a cyclic ester having the formula:

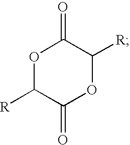

wherein each R is a unit independently chosen from hydrogen, methyl, or ethyl; or
ii) from 1 to 3 equivalents of a lactone having the formula:

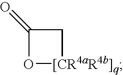

wherein each R^{4a} and R^{4b} is a unit independently chosen from hydrogen, methyl, or ethyl; the index q is from 1 to 7; with 1 equivalent of a polyol having the formula:

HO[(CR^{1a}R^{1b})ₙO]ₘ[CR^{2a}R^{2b}]ⱼOH
each R^{1a}, R^{1b}, R^{2a}, and R^{2b} is independently chosen from hydrogen, methyl, or ethyl; the index j is from 2 to 10; the index m is from 0 to 100; the index n is from 2 to 6; to form an intermediate having the formula:

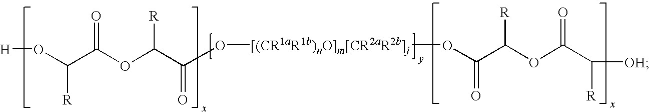

or an intermediate having the formula:

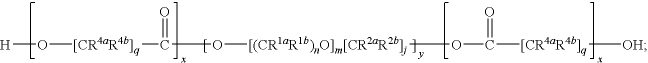

and

b) reacting one of the intermediates formed in step (a) with:
i) a diisocyanate having the formula:

OCN—Z—NCO
Z is a unit comprising one or more:
i) substituted or unsubstituted C₁-C₁₂ linear or branched hydrocarbyl:
ii) substituted or unsubstituted C₃-C₇ carbocyclic rings;
iii) substituted or unsubstituted C₁-C₉ heteroaryl rings;
iv) substituted or unsubstituted C₁-C₉ heterocyclic rings; or
v) substituted or unsubstituted C₆, C₁₀, or C₁₋₄ aryl rings; and
ii) diol having the formula:

HO(CR^{3a}R^{3b})ₖOH
each R^{3a} and R^{3b} is independently chosen from hydrogen, methyl, or ethyl; the index k is from 2 to 10.
In such a way that the ratio of NCO:OH=1-1.1

The following examples describe the preparation of biodegradable polymers according to the present disclosure. As indicated by the examples herein below, the ratio of [Bio] units to [Polyol] units (ratio of x to y) is a choice which the formulator can make when determining the final properties of the biodegradable polymers. Also, the ratio of the [Bio]-[Polyol]-[Bio] pre-polymer diol (x+y) to the L linking units (urethane forming units), and thereby determining the ratio of (x+y) to z, can also be determined by the formulator depending upon the desired final molecular weight and properties of the biodegradable polymers

### Preparation of [Bio][Polyol][Bio] Diols

### EXAMPLE 1

### Copolymer Comprising Polylactide

Step (a) Preparation of terathane-co-(polylactide)

Terathane is dried under vacuum with stirring at 50° C. for 8-10 hrs. The molten terathane is weighed in a dried reaction flask with a stir bar. Lactide, either a mixture of D,L-lactide, or the individual isomers (0.04 moles or 4 equivalents) is added to terathane. The reaction flask is then covered and flushed with N₂. The reaction mixture is then heated to 140° C. with stirring for 1 hour or until all of the lactide has melted. Then temperature is lowered to 110° C. and tin octanoate catalyst (0.02 g) is added using a syringe. The reaction is continued at 110° C. for 48 hrs. At this point it is convenient to analyze the reaction mixture for the presence of unreacted lactide. A sample is taken extracted with a 1:9 dioxane/acetonitrile mixture followed by quantification of the extract by HPLC. The unreacted lactide content can also be quantified by ¹H NMR. If the unreacted lactide content is more than about 2% by weight of the reaction mixture, the reaction can be worked up and purified by sublimation or distillation under vacuum for removal of any untreated lactide. The number average molecular weight of the product is determined by ¹⁹F NMR of the trifluoroacetic ester of the product. This can be accomplished by treating a sample or the dried product (approximately 50 mg) with an excess of trifluoroacetic anhydride (200 μL) in 1 mL of dry dichloromethane. The solvent and excess TFAA are removed first by letting the reaction vials sit inside a well vented hood for 2-8 hours, then drying the sample under high vacuum. Once free of solvent and excess trifluoroacetic acid, the sample is taken up in CDCl₃ and the ¹⁹F NMR spectrum is obtained using trifluorotoluene (10 μL) as an internal standard.

### Preparation of Polyurethane

The dried lactide-terathane pre-polymer (3.2 mmol) is weighed in a dry reaction flask with a stir bar and covered with a septa and heated to 50-60° C. The reaction vessel is the evacuated the flushed with inert gas. This procedure can be repeated until the system is judged to be inert. 1,2 Propanediol (19.7 mmol, 1.50 g) (pre-distilled) is added to the reaction flask using a syringe and the solution is thoroughly mixed. Lysine diisocyanate (24.2 mmol, 5.13 g) is added via a syringe and the reaction is stirred until the solution is homogenous. Tin(II) octanoate is added (0.003 g) via syringe and vortexed to insure good mixing. Immediately after mixing, the reaction solution is subjected to high vacuum until all the bubbles in the reaction mixture have disappeared (˜10 minutes). The temperature of the reaction solution increases during evacuation. Once complete, the reaction flask is flushed with N₂ and cured at 50° C. for 42 hours. After curing, the polyurethane is dissolved in tetrahydrofuran (1:10 by volume) and purified by precipitation with acetate buffer (pH 4.2 20 mM acetate buffer). The precipitated polymer is then lyophilized until fully dry. The resulting polymer is then characterized for MW distribution by gel permeation chromatography (GPC) equipped with a light scattering and refractive index detector using DMF as the eluent and an organic GPC column. The melting range and the glass transition temperature is determined either by differential scanning calorimetry (DSC) or by dynamic rheology at temperature range of 50 to 200° C.

The polyurethane is incorporated with a lubricant (ethylene-bis-stearamide) and a therapeutic drug (optional) by dissolving in tetrahydrofuran and then removing the solvent under vacuum. The resulting polyurethane films are further dried until constant weight and then cut into small pieces. These pieces are then extruded to obtain solid cross-sectional rods. The rods are subjected to tensile testing to measure the Young's modulus.

### EXAMPLE 2

### Step (a) Preparation of terathane-co-(caprolactone)

Terathane is dried under vacuum with stirring at 50° C. for 8-10 hrs. The molten terathane is weighed in a dried reaction flask with a stir bar. Caprolactone (0.04 moles or 4 equivalents) is added to terathane. The reaction flask is then covered and flushed with N₂. The reaction mixture is then heated to 140° C. with stirring for 1 hour or until all of the lactide has melted. Then temperature is lowered to 110° C. and tin octanoate catalyst (0.02 g) is added using a syringe. The reaction is continued at 110° C. for 48 hrs. At this point it is convenient to analyze the reaction mixture for the presence of unreacted lactide. A sample is taken extracted with a 1:9 dioxane/acetonitrile mixture followed by quantification of the extract by HPLC. The unreacted lactide content can also be quantified by ¹H NMR. If the unreacted lactide content is more than about 2% by weight of the reaction mixture, the reaction can be worked up and purified by sublimation or distillation under vacuum for removal of any untreated lactide. The number average molecular weight of the product is determined by ¹⁹F NMR of the trifluoroacetic ester of the product. This can be accomplished by treating a sample or the dried product (approximately 50 mg) with an excess of trifluoroacetic anhydride (200 μL) in 1 mL of dry dichloromethane. The solvent and excess TFAA are removed first by letting the reaction vials sit inside a well vented hood for 2-8 hours, then drying the sample under high vacuum. Once free of solvent and excess trifluoroacetic acid, the sample is taken up in CDCl₃ and the ¹⁹F NMR spectrum is obtained using trifluorotoluene (10 μL) as an internal standard.

### Preparation of Polyurethane

The dried caprolactone-terathane pre-polymer (3.2 mmol) is weighed in a dry reaction flask with a stir bar and covered with a septa and heated to 50-60° C. The reaction vessel is the evacuated the flushed with inert gas. This procedure can be repeated until the system is judged to be inert. 1,2 Propanediol (19.7 mmol, 1.50 g) (pre-distilled) is added to the reaction flask using a syringe and the solution is thoroughly mixed. Lysine diisocyanate (24.2 mmol, 5.13 g) is added via a syringe and the reaction is stirred until the solution is homogenous. Tin(II) octanoate is added (0.003 g) via syringe and vortexed to insure good mixing. Immediately after mixing, the reaction solution is subjected to high vacuum until all the bubbles in the reaction mixture have disappeared (˜10 minutes). The temperature of the reaction solution increases during evacuation. Once complete, the reaction flask is flushed with N₂ and cured at 50° C. for 42 hours. After curing, the polyurethane is dissolved in tetrahydrofuran (1:10 by volume) and purified by precipitation with acetate buffer (pH 4.2 20 mM acetate buffer). The precipitated polymer is then lyophilized until fully dry. The resulting polymer is then characterized for MW distribution by gel permeation chromatography (GPC) equipped with a light scattering and refractive index detector using DMF as the eluent and an organic GPC column. The melting range and the glass transition temperature is determined either by differential scanning calorimetry (DSC) or by dynamic rheology at temperature range of 50 to 200° C.

The polyurethane is incorporated with a lubricant (ethylene-bis-stearamide) and a therapeutic drug (optional) by dissolving in tetrahydrofuran and then removing the solvent under vacuum. The resulting polyurethane films are further dried until constant weight and then cut into small pieces. These pieces are then extruded to obtain solid cross-sectional rods. The rods are subjected to tensile testing to measure the Young's modulus.

### EXAMPLE 3

Preparation of pre-polymer diols derived from α-hydroxy cyclic ester [Bio] unit pre-cursors having the formula:

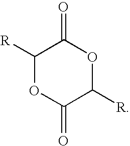

The following general procedure can be used to prepare the pre-polymer diols according to the present disclosure (See Choi et al, J. Biomater. Sci., Polym. Edn., 13 (10), 1163-1173, 2002 included herein in its entirety by reference).

A [Polyol] unit precursor monomer, for example, polytetramethylene ether glycol (polyterathane) having an average molecular weight of about 2000 g/mol and lactide (R equal to methyl) are charged to a round bottom flask and the mixture is heated to 140° C. until the contents are fully melted. The flask is evacuated several times and purged with nitrogen until and inert atmosphere is achieved. The temperature is then lowered to 120° C. and tin octoate (0.001 mol equivalent based upon the amount of lactide used for the formation of the pre-polymer diol). The reaction is allowed to proceed for 24 hours under the inert atmosphere of nitrogen. Once complete, the product is dissolved in a first solvent, (for the combination of polyterathane and lactide, toluene is a convenient first solvent) and then precipitated using a second solvent, inter alia, hexane. The solvent is then removed and the polymer dried under vacuum.

Table I herein below provides non-limiting examples of pre-polymer diols which are precursors to the biodegradable polymers according to the present disclosure.

TABLE I

| | Equiv. | Equiv. of | Average | % | |
|---|---|---|---|---|---|
| No. | terathane¹ | lactide² | MW of diol³ | yield | Ratio⁴ |
| 1 | 1 | 6 | 2860 | 60.5 | 1:6 |
| 2 | 1 | 12 | 3440 | 72.0 | 1:10 |

¹Terathane having an average molecular weight of about 2000 g/mol.
²Lactide; cyclic ester wherein each R unit is methyl.
³Molecular weight determination by ¹H NMR.
⁴Final ratio of terathane derived [Polyol] units to lactide derived [Bio] units in the final pre-polymer diol.

The pre-polymer diol No. 1 from Table I can be represented by the formula:

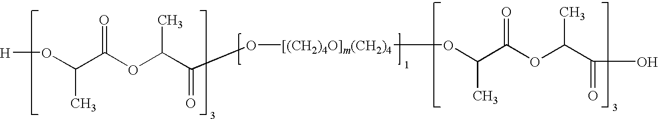

wherein the index m has an average value of about 26.5.

### EXAMPLE 4

Utilizing the same procedure as described herein above in Example 1, pre-polymer diols derived from cyclic lactones having the formula:

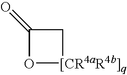

wherein each R^{4a} and R^{4b} is a unit independently chosen from hydrogen, methyl, or ethyl; the index q is from 1 to 7; can be prepared. A non-limiting example of a suitable lactone is caprolactone.

Table II herein below provides non-limiting examples of pre-polymer diols which were formed according to Example 2.

TABLE II

| | Equiv. | | Average | % | |
|---|---|---|---|---|---|
| No. | terathane | Equiv. of lactone¹ | MW of diol² | yield | Ratio³ |
| 3 | 1 | 6 | 2690 | 83.1 | 1:6 |
| 4 | 1 | 12 | 3370 | 79.7 | 1:12 |

¹Caprolactone.
²Molecular weight determination by ¹H NMR.
³Final ratio of caprolactone derived [Polyol] units to lactide derived [Bio] units in the final pre-polymer diol.

The pre-polymer diol No. 3 from Table II can be represented by the formula:

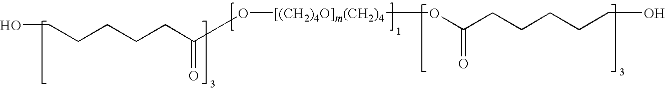

wherein the index m has an average value of about 26.5.

### EXAMPLE 5

The pre-polymer diols of the present disclosure are reacted with a diisocyanate as described herein above, utilizing any of the two reaction conditions described herein below.

### a) Melt Conditions

Prior to use, the pre-polymer diols are typically dried under high vacuum for at least 4 days. The reaction can be carried out as described in Gorna K, Polowinski S, Gogolewski S, J. Polym. Sci., Part A Polym. Chem., 40, 156-170, 2002, included herein by reference in its entirety. A round bottom flask equipped with an efficient stirrer and reflux condenser is charged with a pre-polymer diol and heated to about 60° C. until melted and held at that temperature under a stream of dry nitrogen gas for about 4 hours. The temperature is lowered to between 50° C. and 55° C. A catalyst is added then the diisocyanate. The temperature is then raised to about 60° C. and the reaction mixture stirred for about 2 hours. At this point any chain extender is added and the stirring continued until the increase in viscosity prohibits further mixing. The temperature is reduce to about 25° C. and the crude polymer is held overnight under nitrogen atmosphere. The next day the crude polymer is dissolved in dimethylformamide and precipitated by the addition of water. The isolated polymer is thoroughly dried under vacuum prior to fabrication into an intravaginal ring.

### b) Solution Conditions

Prior to use, the pre-polymer diols are typically dried under high vacuum for at least 4 days. The reaction can be carried out as described in Woodhouse K A, Skarja, G A, J. Appl. Polym. Sci., 75, 1522-34, 2000, included herein by reference in its entirety. A round bottom flask equipped with an efficient stirrer and reflux condenser is charged with a pre-polymer diol in dimethylformamide (DMF) at about 70° C. The flask is evacuated and flushed with nitrogen. This is repeated twice more. The desired diisocyanate and catalyst are added and stirring is continued for 2.5 hours after which the temperature of the reaction is lowered to room temperature and the chain extender is added. The reaction is allowed to continue for 18 hours. Water is then added to precipitate the final biodegradable polyurethane polymer which is then dried under high vacuum before fabrication into an intervaginal ring.

TABLE III

| | | Equiv. of | Equiv. | % | |
|---|---|---|---|---|---|
| No. | diol | diol | MBCD¹ | yield | method |
| 5 | 1 | 1.0 | 2.1 | 70.5 | melt |
| 6 | 2 | 1.0 | 2.1 | 68.5 | melt |
| 7 | 1 | 1.0 | 2.1 | 95.0 | solution² |
| 8 | 2 | 1.0 | 2.1 | 98.6 | solution |
| 9 | 3 | 1.0 | 2.1 | 86.2 | solution |
| 10 | 4 | 1.0 | 2.1 | 89.5 | solution |

¹MBCD = methylene bis(cyclohexyldiisocyanate).
²Solvent is DMF.

The following are further non-limiting examples of biodegradable polymers according to the present disclosure:

Biodegradable polymers formed from pre-polymer diols comprising lactide and polytetramethylene glycols (terathanes) and diisocyanate derived urethane and propylene glycol chain extender having the formula:

TABLE IV

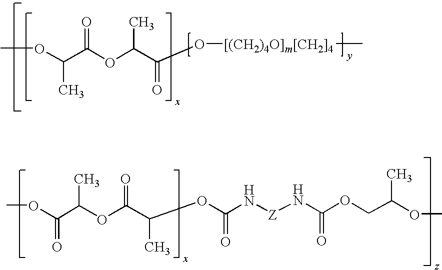

| No. | Z | m³ | x | y | z |
|---|---|---|---|---|---|
| 11 | bis(cyclohex-4yl)methane¹ | 13 | 1 | 1 | 1 |
| 12 | bis(cyclohex-4-yl)methane | 20 | 1 | 1 | 1 |
| 13 | bis(cyclohex-4-yl)methane | 27 | 1 | 1 | 1 |
| 14 | bis(cyclohex-4-yl)methane | 13 | 1.5 | 1 | 1 |
| 15 | bis(cyclohex-4-yl)methane | 20 | 1.5 | 1 | 1 |
| 16 | bis(cyclohex-4-yl)methane | 27 | 1.5 | 1 | 1 |
| 17 | bis(cyclohex-4-yl)methane | 13 | 2 | 1 | 1 |
| 18 | bis(cyclohex-4-yl)methane | 20 | 2 | 1 | 1 |
| 19 | bis(cyclohex-4-yl)methane | 27 | 2 | 1 | 1 |
| 20 | bis(cyclohex-4-yl)methane | 13 | 25 | 1 | 1 |
| 21 | bis(cyclohex-4-yl)methane | 20 | 25 | 1 | 1 |
| 22 | bis(cyclohex-4-yl)methane | 27 | 25 | 1 | 1 |
| 23 | bis(cyclohex-4-yl)methane | 13 | 3 | 1 | 1 |
| 24 | bis(cyclohex-4-yl)methane | 20 | 3 | 1 | 1 |
| 25 | bis(cyclohex-4-yl)methane | 27 | 3 | 1 | 1 |
| 26 | bis(cyclohex-4-yl)methane | 13 | 3 | 1 | 1 |
| 27 | bis(cyclohex-4-yl)methane | 20 | 1 | 1 | 2 |
| 28 | bis(cyclohex-4-yl)methane | 27 | 1 | 1 | 2 |
| 29 | bis(cyclohex-4-yl)methane | 13 | 1 | 1 | 2 |
| 30 | bis(cyclohex-4-yl)methane | 20 | 1.5 | 1 | 2 |
| 31 | bis(cyclohex-4-yl)methane | 27 | 1.5 | 1 | 2 |
| 32 | bis(cyclohex-4-yl)methane | 13 | 1.5 | 1 | 2 |
| 33 | bis(cyclohex-4-yl)methane | 20 | 2 | 1 | 2 |
| 34 | bis(cyclohex-4-yl)methane | 27 | 2 | 1 | 2 |
| 35 | bis(cyclohex-4-yl)methane | 13 | 2 | 1 | 2 |
| 36 | bis(cyclohex-4-yl)methane | 20 | 25 | 1 | 2 |
| 37 | bis(cyclohex-4-yl)methane | 27 | 25 | 1 | 2 |
| 38 | bis(cyclohex-4-yl)methane | 13 | 25 | 1 | 2 |
| 39 | bis(cyclohex-4-yl)methane | 20 | 3 | 1 | 2 |
| 40 | bis(cyclohex-4-yl)methane | 27 | 3 | 1 | 2 |
| 41 | bis(cyclohex-4-yl)methane | 13 | 3 | 1 | 2 |
| 42 | bis(cyclohex-4-yl)methane | 20 | 3 | 1 | 2 |
| 43 | 1-carboxypentylene² | 27 | 1 | 1 | 1 |
| 44 | 1-carboxypentylene | 13 | 1 | 1 | 1 |
| 45 | 1-carboxypentylene | 20 | 1 | 1 | 1 |
| 46 | 1-carboxypentylene | 27 | 1.5 | 1 | 1 |
| 47 | 1-carboxypentylene | 13 | 1.5 | 1 | 1 |
| 48 | 1-carboxypentylene | 20 | 1.5 | 1 | 1 |
| 49 | 1-carboxypentylene | 27 | 2 | 1 | 1 |
| 50 | 1-carboxypentylene | 13 | 2 | 1 | 1 |
| 51 | 1-carboxypentylene | 20 | 2 | 1 | 1 |
| 52 | 1-carboxypentylene | 27 | 25 | 1 | 1 |
| 53 | 1-carboxypentylene | 13 | 25 | 1 | 1 |
| 54 | 1-carboxypentylene | 20 | 25 | 1 | 1 |
| 55 | 1-carboxypentylene | 27 | 3 | 1 | 1 |
| 56 | 1-carboxypentylene | 13 | 3 | 1 | 1 |
| 57 | 1-carboxypentylene | 20 | 3 | 1 | 1 |
| 58 | 1-carboxypentylene | 27 | 1 | 1 | 1 |
| 59 | 1-carboxypentylene | 13 | 1 | 1 | 2 |
| 60 | 1-carboxypentylene | 20 | 1 | 1 | 2 |
| 61 | 1-carboxypentylene | 27 | 1.5 | 1 | 2 |
| 62 | 1-carboxypentylene | 13 | 1.5 | 1 | 2 |
| 63 | 1-carboxypentylene | 20 | 1.5 | 1 | 2 |
| 64 | 1-carboxypentylene | 27 | 2 | 1 | 2 |
| 65 | 1-carboxypentylene | 13 | 2 | 1 | 2 |
| 66 | 1-carboxypentylene | 20 | 2 | 1 | 2 |
| 67 | 1-carboxypentylene | 27 | 25 | 1 | 2 |
| 68 | 1-carboxypentylene | 13 | 25 | 1 | 2 |
| 69 | 1-carboxypentylene | 20 | 25 | 1 | 2 |
| 70 | 1-carboxypentylene | 27 | 3 | 1 | 2 |
| 71 | 1-carboxypentylene | 13 | 3 | 1 | 2 |
| 72 | 1-carboxypentylene | 20 | 3 | 1 | 2 |

¹L unit derived from methylene bis(cyclohexyldiisocyanate).
²L unit derived from lysine diisocyanate
³This represents a approximate whole number of the average value of the index m, for example, a poly(tetramethylene) glycol having an average molecular weight of 1000 g/mol would have an average index of m of approximately 12.6.

Biodegradable polymers formed from pre-polymer diols comprising caprolactone and polytetramethylene glycols (terathanes) and a diisocyanate derived urethane and propylene glycol chain extender having the formula:

TABLE V

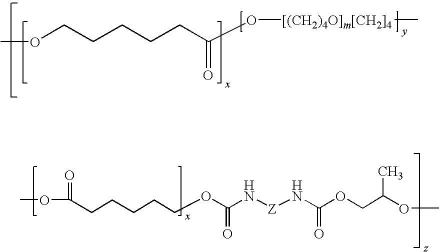

| No. | Z | m³ | x | y | z |
|---|---|---|---|---|---|
| 73 | bis(cyclohex-4yl)methane¹ | 13 | 1 | 1 | 1 |
| 74 | bis(cyclohex-4-yl)methane | 20 | 1 | 1 | 1 |
| 75 | bis(cyclohex-4-yl)methane | 27 | 1 | 1 | 1 |
| 76 | bis(cyclohex-4-yl)methane | 13 | 1.5 | 1 | 1 |
| 77 | bis(cyclohex-4-yl)methane | 20 | 1.5 | 1 | 1 |
| 78 | bis(cyclohex-4-yl)methane | 27 | 1.5 | 1 | 1 |
| 79 | bis(cyclohex-4-yl)methane | 13 | 2 | 1 | 1 |
| 80 | bis(cyclohex-4-yl)methane | 20 | 2 | 1 | 1 |
| 81 | bis(cyclohex-4-yl)methane | 27 | 2 | 1 | 1 |
| 82 | bis(cyclohex-4-yl)methane | 13 | 25 | 1 | 1 |
| 83 | bis(cyclohex-4-yl)methane | 20 | 25 | 1 | 1 |
| 84 | bis(cyclohex-4-yl)methane | 27 | 25 | 1 | 1 |
| 85 | bis(cyclohex-4-yl)methane | 13 | 3 | 1 | 1 |
| 86 | bis(cyclohex-4-yl)methane | 20 | 3 | 1 | 1 |
| 87 | bis(cyclohex-4-yl)methane | 27 | 3 | 1 | 1 |
| 88 | bis(cyclohex-4-yl)methane | 13 | 3 | 1 | 1 |
| 89 | bis(cyclohex-4-yl)methane | 20 | 1 | 1 | 2 |
| 90 | bis(cyclohex-4-yl)methane | 27 | 1 | 1 | 2 |
| 91 | bis(cyclohex-4-yl)methane | 13 | 1 | 1 | 2 |
| 92 | bis(cyclohex-4-yl)methane | 20 | 1.5 | 1 | 2 |
| 93 | bis(cyclohex-4-yl)methane | 27 | 1.5 | 1 | 2 |
| 94 | bis(cyclohex-4-yl)methane | 13 | 1.5 | 1 | 2 |
| 95 | bis(cyclohex-4-yl)methane | 20 | 2 | 1 | 2 |
| 96 | bis(cyclohex-4-yl)methane | 27 | 2 | 1 | 2 |
| 97 | bis(cyclohex-4-yl)methane | 13 | 2 | 1 | 2 |
| 98 | bis(cyclohex-4-yl)methane | 20 | 25 | 1 | 2 |
| 99 | bis(cyclohex-4-yl)methane | 27 | 25 | 1 | 2 |
| 100 | bis(cyclohex-4-yl)methane | 13 | 25 | 1 | 2 |
| 101 | bis(cyclohex-4-yl)methane | 20 | 3 | 1 | 2 |
| 102 | bis(cyclohex-4-yl)methane | 27 | 3 | 1 | 2 |
| 103 | bis(cyclohex-4-yl)methane | 13 | 3 | 1 | 2 |
| 104 | bis(cyclohex-4-yl)methane | 20 | 3 | 1 | 2 |
| 105 | 1-carboxypentylene² | 27 | 1 | 1 | 1 |
| 106 | 1-carboxypentylene | 13 | 1 | 1 | 1 |
| 107 | 1-carboxypentylene | 20 | 1 | 1 | 1 |
| 108 | 1-carboxypentylene | 27 | 1.5 | 1 | 1 |
| 109 | 1-carboxypentylene | 13 | 1.5 | 1 | 1 |
| 110 | 1-carboxypentylene | 20 | 1.5 | 1 | 1 |
| 111 | 1-carboxypentylene | 27 | 2 | 1 | 1 |
| 112 | 1-carboxypentylene | 13 | 2 | 1 | 1 |
| 113 | 1-carboxypentylene | 20 | 2 | 1 | 1 |
| 114 | 1-carboxypentylene | 27 | 25 | 1 | 1 |
| 115 | 1-carboxypentylene | 13 | 25 | 1 | 1 |
| 116 | 1-carboxypentylene | 20 | 25 | 1 | 1 |
| 117 | 1-carboxypentylene | 27 | 3 | 1 | 1 |
| 118 | 1-carboxypentylene | 13 | 3 | 1 | 1 |
| 119 | 1-carboxypentylene | 20 | 3 | 1 | 1 |
| 120 | 1-carboxypentylene | 27 | 1 | 1 | 1 |
| 121 | 1-carboxypentylene | 13 | 1 | 1 | 2 |
| 122 | 1-carboxypentylene | 20 | 1 | 1 | 2 |
| 123 | 1-carboxypentylene | 27 | 1.5 | 1 | 2 |
| 124 | 1-carboxypentylene | 13 | 1.5 | 1 | 2 |
| 125 | 1-carboxypentylene | 20 | 1.5 | 1 | 2 |
| 126 | 1-carboxypentylene | 27 | 2 | 1 | 2 |
| 127 | 1-carboxypentylene | 13 | 2 | 1 | 2 |
| 128 | 1-carboxypentylene | 20 | 2 | 1 | 2 |
| 129 | 1-carboxypentylene | 27 | 25 | 1 | 2 |
| 130 | 1-carboxypentylene | 13 | 25 | 1 | 2 |
| 131 | 1-carboxypentylene | 20 | 25 | 1 | 2 |
| 132 | 1-carboxypentylene | 27 | 3 | 1 | 2 |
| 133 | 1-carboxypentylene | 13 | 3 | 1 | 2 |
| 134 | 1-carboxypentylene | 20 | 3 | 1 | 2 |

¹L unit derived from methylene bis(cyclohexyldiisocyanate).
²L unit derived from lysine diisocyanate
³This represents a approximate whole number of the average value of the index m, for example, a poly(tetramethylene) glycol having an average molecular weight of 1000 g/mol would have an average index of m of approximately 12.6.

### EXAMPLE 6

### Preparation of Polyurethane

The dried caprolactone-terathane pre-polymer (3.1 mmol) is weighed in a dry reaction flask with a stir bar and covered with a septa and heated to 50-60° C. The reaction vessel is the evacuated the flushed with inert gas. This procedure can be repeated until the system is judged to be inert. 1,4 butanediol (12.9 mmol) (pre-distilled) is added to the reaction flask using a syringe and the solution is thoroughly mixed. 4,4′-methylene-bis-cyclohexane diisocyanate (15.9 mmol) is added via a syringe and the reaction is stirred until the solution is homogenous. Tin(II) octanoate is added (0.002 g) via syringe and vortexed to insure good mixing. Immediately after mixing, the reaction solution is subjected to high vacuum until all the bubbles in the reaction mixture have disappeared (˜10 minutes). The temperature of the reaction solution increases during evacuation. Once complete, the reaction flask is flushed with N₂ and cured at 110° C. for 4-5 hours. After curing, the polyurethane is dissolved in tetrahydrofuran (1:10 by volume) and purified by precipitation with acetate buffer (pH 4.2 20 mM acetate buffer). The precipitated polymer is then lyophilized until fully dry. The resulting polymer is then characterized for MW distribution by gel permeation chromatography (GPC) equipped with a light scattering and refractive index detector using DMF as the eluent and an organic GPC column. The melting range and the glass transition temperature is determined either by differential scanning calorimetry (DSC) or by dynamic rheology at temperature range of 50 to 200° C.

The polyurethane is incorporated with a lubricant (ethylene-bis-stearamide) and a therapeutic drug (optional) by dissolving in tetrahydrofuran and then removing the solvent under vacuum. The resulting polyurethane films are further dried until constant weight and then cut into small pieces. These pieces are then extruded to obtain solid cross-sectional rods. The rods are subjected to tensile testing to measure the Young's modulus.

### Intravaginal Devices

The biodegradable/biocompatible copolymers disclosed herein can provide for one or more unmet medical needs, including:
A) providing a means for delivering a antiviral agent, inter alia, a therapeutic agent effective against Human Immunodeficiency Virus (HIV), for example, anti-viral agents which are not deliverable to a patient orally;
B) providing a means for delivery of a combination of therapeutic agents and/or prophylactic agents and/or contraceptive agents wherein the first agent is delivered for a first period of time, after which a second agent is delivered; and
C) providing a means for delivering a mixture of therapeutic agents, including:
   i) birth control hormones and/or spermicidal agents and/or other contraceptive agents;
   ii) anti-HIV mucosal vaccines;
   iii) post-menopausal hormones;
   iv) pharmaceuticals for treatment of sexually transmitted diseases (STD's) including HPV, HSV;
   v) therapeutic peptides and proteins;
   vi) pharmaceuticals for the treatment of vaginal infections such as bacterial vaginosis, and yeast infections
   vii) pharmaceuticals for the treatment of diseases such as arthritis, endometriosis, diabetes, and polycystic ovarian symptoms

Each of the above medical needs is met without the need for inspection or removal of the intravaginal device and after the use of the intravaginal device, the device can be discarded and will subsequently biodegrade.

One aspect of the intravaginal devices relates to delivery of antiviral agents. The following are non-limiting examples of antiviral agents: Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; BMS 806; C31G; Carageenan; Cellulose sulfate; Cyclodextrins; Dapivirine; Delavirdine Mesylate; Desciclovir; Dextrin 2-sulfate; Didanosine; Disoxaril; Edoxudine; Enviradene; Enviroxime; Etravirine; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Merck 167; Methisazone; Nevirapine; PCS-Rantes; Penciclovir; Pirodavir; Ribavirin; Rimantadine Hydrochloride; Rilpivirine (TMC-278); Saquinavir Mesylate; SCH-C; SCH-D; Somantadine Hydrochloride; Sorivudine; Statolon; Stavudine; T20; Tilorone Hydrochloride; TMC120; TMC125; Trifluridine; Tenofovir; UC-781; UK-427; UK-857; Valacyclovir Hydrochloride; Vidarabine; Vidarabine Phosphate; Vidarabine Sodium Phosphate; Viroxime; Zalcitabine; Zidovudine; Zinviroxime.

In one embodiment the therapeutic agent is a non-nucleoside reverse transcriptase inhibitor, a non-limiting example of which is 4-[4-(mesitylamino)pyrimidin-2-ylamino)-benzonitrile (TMC-120) having the formula:

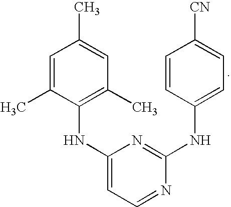

In another embodiment, the intravaginal devices disclosed herein can deliver hormonal contraceptives. Non-limiting examples of contraceptives include Leveongestrel, nestorone, 17a-ethinyl-levongestrel-17b-hydroxy-estra-4,9,11-trien-3-one, norethindrone, norgestrienono, and estradiol.

The following are the non-limiting examples of other therapeutic agents and their corresponding medical indication.

| Agent | Medical Indication |
|---|---|
| Sildenafil | Increased bloodflow to the uterus in preparation for |
| | embryo implantation |
| Misoprostol | Induction of labor, cervical ripening, pregnancy |
| | termination |
| Oxybutynin | Overactive bladder |
| Indomethacin | Treatment of preterm labor |
| Bromocriptine | Treatment of prolactinoma for intolerance of nausea/ |
| | vomiting side effects |
| Lidocaine | Cervical anastetic |

### Intravaginal Rings

The biodegradable polyurethanes described herein above provide a wide range of flexibility to the formulator. The one aspect of the disclosed intravaginal devices relates to intravaginal rings that can have any shape, configuration, or size desirable. For example, in a first embodiment of the present disclosure relates to a circular ring comprising a therapeutic agent homogeneously dispersed therein.

### Intravaginal Ring Fabrication

The following are non-limiting examples of procedures for fabricating the intravaginal rings of the present disclosure.

A first embodiment of the present disclosure which relates to a single therapeutic agent dispersed therein can be prepared as follows. The polymer and the therapeutic agent are dissolved in a common solvent after which the solvent is removed. The solvent can be removed by any means available to the formulator, inter alia, under reduced pressure or by lyophilization.

Once the polymer/therapeutic agent material is obtained, the ring may be formed by extruding the therapeutic agent containing biodegradable polyurethane, cutting the extruded material to a desired length, then attaching the two ends to form a ring. The two ends of the material can be attached by any means available to the formulator, for example, by heating the two ends and allowing the material to cool, by applying an amount of a molten compatible biodegradable adhesive, or by applying an amount of molten biodegradable polyurethane from which the ring is formed.

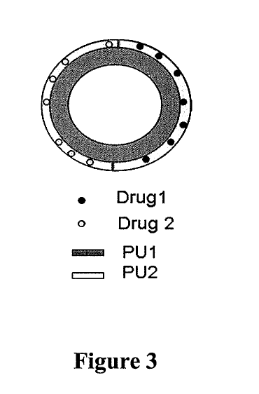

A further embodiment of the present disclosure relates to rings having a first segment and a second segment, wherein dispersed within the first segment is a first therapeutic agent, for example, a drug, and a second therapeutic, for example a different drug, dispersed within the second segment. The two segments can be made by dispersing each of the therapeutic agents in separate batches of a biodegradable polyurethane then extruding the drug loaded polymer as described herein above. The two segments can also be attached to one another by any means available to the formulator. This embodiment is depicted in FIG. 3.

In an iteration of this embodiment, the two therapeutic agent containing segments can be joined by segments which do not comprise a therapeutic agent, or which comprise a material which aids in the absorption of the therapeutic agent. Also the non-therapeutic agent can be material which provides a benefit, for example, a vitamin, a vaginal lubricant, or an agent with facilitates the maintenance of the ring in position within the vagina.

In yet another embodiment, the ring can consist of a core comprising a first biodegradable polyurethane having no therapeutic agent dispersed therein, covered by a layer of the same or different biodegradable polyurethane which does have dispersed therein one or more therapeutic agents. The second biodegradable polyurethane can have properties which are optimized by selecting the right ratio of the [Bio] unit to other polymer units, thereby achieving the desired degradation rate in vivo which allows the formulator to achieve a tailored release kinetics for the incorporated therapeutic agent. In this embodiment it is envisioned the first biodegradable polyurethane (the layer not comprising a therapeutic agent) will have a slower rate of biodegradation and therefore serve as a support for the segment which is releasing the therapeutic agent. This embodiment relates to enrobing a non-therapeutic segment of biodegradable polymer with a layer of biodegradable polymer having a therapeutic agent.

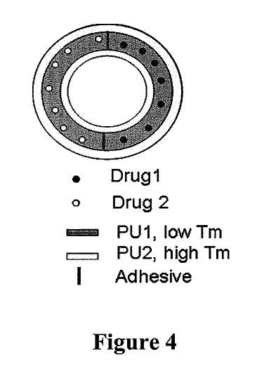

Rings according to this embodiment may be fabricated by first extruding a tubular segment of biodegradable polyurethane which comprises a deliverable therapeutic agent after which the hollow portion is filled in any manner available to the formulator with the second, slower biodegrading polymer not comprising a therapeutic agent. This embodiment is depicted in FIG. 4.

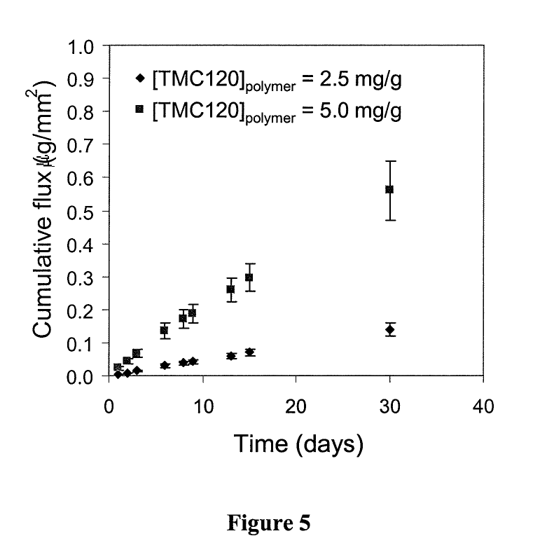

The present disclosure also allows for the delivery of temperature sensitive therapeutic agents. For example, the extrusion of the temperature sensitive polyurethane will can be done a lower temperature by modifying the units which comprise the biodegradable polymer, however, lower extrusion temperatures can be achieved by adding a biocompatible plasticizer rather than modifying the polymer itself. Again, a hollow tube composed of a higher melting biocompatible polyurethane can be extruded, followed by filling the core with the drug-loaded lower melting polyurethane. In an alternative iteration, the higher melting outer layer can be filled with a therapeutic agent which is dispersed in an excipient gel. This latter iteration is especially useful for very highly temperature sensitive actives. An example of this embodiment is depicted in FIG. 5. This example also relates to enrobing a core biodegradable polymer with a second outer layer of biodegradable polymer.

A non-limiting example of the present disclosure is a medical device comprising as at least one of the biodegradable polymers a polymer having the formula:

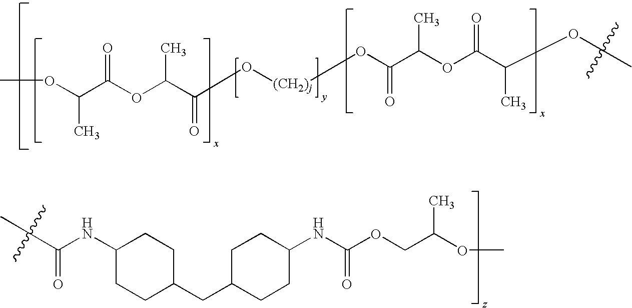

wherein j is from 2 to 6; the ratio of the index x to y is from 1.5:1 to 2.5:1; the index z is from 2 to 5.

A further non-limiting example of the present disclosure is a medical device comprising as at least one of the biodegradable polymers a polymer having the formula:

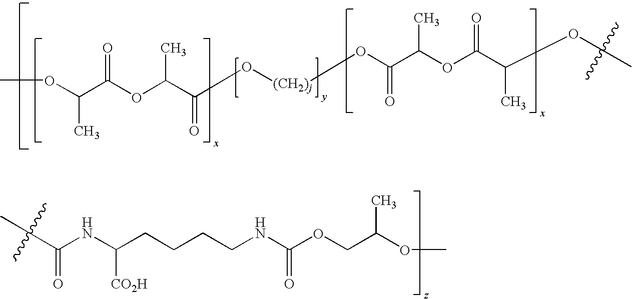

wherein j is from 2 to 6; the ratio of the index x to y is from 1.5:1 to 2.5:1; the index z is from 2 to 5.

### Methods of Treatment and Uses of the Devices

The present disclosure relates to methods for treating one or more diseases utilizing the biodegradable polyurethane intravaginal medical devices described herein above.

Non-limiting examples of methods for treating diseases according to the present disclosure include:

A method for treating or preventing a sexually transmitted disease comprising providing to a patient in need of treatment or in need of prevention of a sexually transmitted disease a medical device according to the present disclosure. This method is especially useful for the treatment of HIV. In addition, the present disclosure is especially useful for delivering a therapeutic agent which is a non-nucleoside reverse transcriptase inhibitor, inter alia, TMC 120 as described herein above.

A method for treating or preventing a sexually transmitted disease comprising providing to a patient in need of treatment or in need of prevention of a sexually transmitted disease an intravaginal ring capable of delivering one or more therapeutic agents. This method is especially useful for the treatment of HIV. In addition, the present disclosure is especially useful for delivering a therapeutic agent which is a non-nucleoside reverse transcriptase inhibitor, inter alia, TMC 120 as described herein above.

A method for treating or preventing a disease comprising providing to a patient in need of treatment or in need of prevention of a disease an intravaginal ring formed by two concentric rings, an outer ring and an inner ring, the two rings in register which each other; wherein the outer ring comprises at least an effective amount of the therapeutic agent. This method is especially useful for the treatment of HIV. In addition, the present disclosure is especially useful for delivering a therapeutic agent which is a non-nucleoside reverse transcriptase inhibitor, inter alia, TMC 120 as described herein above.

A method for treating or preventing a disease comprising providing to a patient in need of treatment or in need of prevention of a disease an intravaginal ring wherein a first biodegradable polymer layer comprising a therapeutic agent enrobes a second biodegradable polymer core comprising no therapeutic agent. This method is especially useful for the treatment of HIV. In addition, the present disclosure is especially useful for delivering a therapeutic agent which is a non-nucleoside reverse transcriptase inhibitor, inter alia, TMC 120 as described herein above.

A method for treating or preventing a disease comprising providing to a patient in need of treatment or in need of prevention of a disease an intravaginal ring wherein a first biodegradable polymer layer comprising a therapeutic agent enrobes a second biodegradable polymer core comprising no therapeutic agent wherein the first polymer layer of the intravaginal ring has a higher melting point than the polymer core. This method is especially useful for the treatment of HIV. In addition, the present disclosure is especially useful for delivering a therapeutic agent which is a non-nucleoside reverse transcriptase inhibitor, inter alia, TMC 120 as described herein above.

A method for treating or preventing a disease comprising providing to a patient in need of treatment or in need of prevention of a disease an intravaginal ring wherein
i) the therapeutic agent is dispersed within the first segment of the ring;
ii) the second therapeutic agent is dispersed within the second segment of the ring; and
ii) the ring is capable of releasing into a person using the medical device an effective amount of the therapeutic agent and the second therapeutic agent for at least about 30 days.
   This method is especially useful for the treatment of HIV. In addition, the present disclosure is especially useful for delivering a therapeutic agent which is a non-nucleoside reverse transcriptase inhibitor, inter alia, TMC 120 as described herein above.

The present disclosure relates to methods for delivering a pharmaceutical composition which inhibits fertilization of an ovum, said method utilizing the biodegradable polyurethane intravaginal medical devices described herein above. A non-limiting example of this comprises:

Providing to a woman who desires not to be impregnated, an intravaginal ring wherein
i) at least one therapeutic agent which is effective for inhibiting the fertilization of an ovum, said therapeutic agent is dispersed within the first segment of the ring;
ii) and optionally, one or more second therapeutic agents dispersed within the second segment of the ring; and
ii) the ring is capable of releasing into a person using the intravaginal ring an effective amount of the therapeutic agent and the second therapeutic agent for at least about 30 days.

The present disclosure further relates to the use of the disclosed copolymers for making a medicament. The following are non-limiting examples.

The use of the disclosed biodegradable and/or biocompatible polymers for making a medicament for treating a sexually transmitted disease comprising providing to a patient in need of treatment or in need of prevention of a sexually transmitted disease a medical device according to the present disclosure.

The use of the disclosed biodegradable and/or biocompatible polymers for making a medicament for preventing a sexually transmitted disease comprising providing to a patient in need of treatment or in need of prevention of a sexually transmitted disease a medical device according to the present disclosure.

The use of the disclosed biodegradable and/or biocompatible polymers for making a medicament for treating of HIV.

The use of the disclosed biodegradable and/or biocompatible polymers for making a medicament for delivering a non-nucleoside reverse transcriptase inhibitor.

The use of the disclosed biodegradable and/or biocompatible polymers for making a medicament for delivering 4-[4-(mesitylamino)pyrimidin-2-ylamino)-benzonitrile.

The use of the disclosed biodegradable and/or biocompatible polymers for making a medical device formed by two concentric rings, an outer ring and an inner ring, the two rings in register which each other; wherein the outer ring comprises at least an effective amount of the therapeutic agent, the medical device useful for preventing conception in a female.

The use of the disclosed biodegradable and/or biocompatible polymers for making a medical device formed by two concentric rings, an outer ring and an inner ring, the two rings in register which each other; wherein the outer ring comprises at least an effective amount of the therapeutic agent, the medical device useful for preventing transmission of a sexually transmitted disease.

The use of the disclosed biodegradable and/or biocompatible polymers for making a medical device formed by two concentric rings, an outer ring and an inner ring, the two rings in register which each other; wherein the outer ring comprises at least an effective amount of the therapeutic agent, the medical device useful for delivering a pharmaceutically active agent.

The use of the disclosed biodegradable and/or biocompatible polymers for making a medical device having a first biodegradable polymer layer comprising a therapeutic agent enrobes a second biodegradable polymer core comprising no therapeutic agent, the medical device useful for preventing conception in a female.

The use of the disclosed biodegradable and/or biocompatible polymers for making a medical device having a first biodegradable polymer layer comprising a therapeutic agent enrobes a second biodegradable polymer core comprising no therapeutic agent, the medical device useful for preventing transmission of a sexually transmitted disease.

The use of the disclosed biodegradable and/or biocompatible polymers for making a medical device having a first biodegradable polymer layer comprising a therapeutic agent enrobes a second biodegradable polymer core comprising no therapeutic agent, the medical device useful for delivering a pharmaceutically active agent.

The use of an intravaginal device as disclosed herein for treating a patient in need of treatment or in need of prevention of a disease wherein:
i) the therapeutic agent is dispersed within the first segment of the ring;
ii) the second therapeutic agent is dispersed within the second segment of the ring; and
ii) the ring is capable of releasing into a person using the medical device an effective amount of the therapeutic agent and the second therapeutic agent for at least about 30 days.

The present disclosure further relates to the use of biocompatible and/or biodegradable copolymers for making a medicament for preventing pregnancy wherein, device comprises:
i) at least one therapeutic agent which is effective for inhibiting the fertilization of an ovum, said therapeutic agent is dispersed within the first segment of the ring;
ii) and optionally, one or more second therapeutic agents dispersed within the second segment of the ring; and
ii) the ring is capable of releasing into a person using the intravaginal ring an effective amount of the therapeutic agent and the second therapeutic agent for at least about 30 days.

The present disclosure further relates to a medical device comprising one or more biocompatible and/or biodegradable copolymers as disclosed herein.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure. All cited references are included herein by reference in their entirety.

What is claimed is:

1. An intravaginal medical device comprising:
a) a biodegradable polymer; and
b) one or more therapeutic and/or prophylactic and/or contraceptive agent;
wherein the medical device is substantially non-biodegradable when in the human body.

2. A medical device according to claim 1 wherein the biodegradable polymer is a polyurethane having the formula: 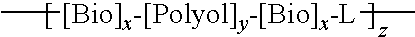
wherein:
Bio is a residue having the formula; 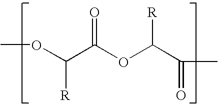
each R is a unit independently chosen from hydrogen, methyl, or ethyl;
Polyol is a residue having the formula: [symbol]
O—[(CR^{1a}R^{1b})ₙO]ₘ—[CR^{2a}R^{2b}]ⱼ [symbol]
each R^{1a}, R^{1b}, R^{2a}, and R^{2b} is independently chosen from hydrogen, methyl, or ethyl; the index j is from 2 to 10; the index m is from 0 to 100; the index n is from 2 to 6; and
L is a linking residue having the formula: 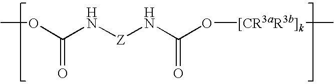
each R^{3a} and R^{3b} is independently chosen from hydrogen, methyl, or ethyl;
Z is a unit comprising one or more:
i) substituted or unsubstituted C₁-C₁₂ linear or branched hydrocarbyl:
ii) substituted or unsubstituted C₃-C₇ carbocyclic rings;
iii) substituted or unsubstituted C₁-C₉ heteroaryl rings;
iv) substituted or unsubstituted C₁-C₉ heterocyclic rings; or
v) substituted or unsubstituted C₆, C₁₀, or C₁₋₄ aryl rings;
the index k is from 2 to 10;
the index x is from 1 to 12; the index y is from 1 to 100; the index z is from 2 to 50.

3. A medical device according to claim 2 wherein the biodegradable polymer has the formula: 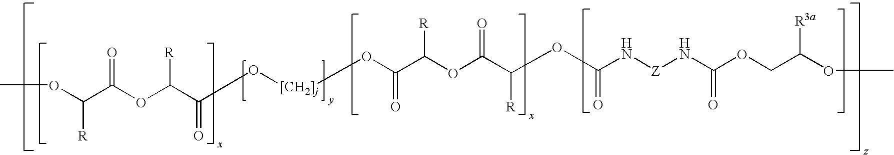
wherein R is chosen from hydrogen or methyl;
R^{3a} is hydrogen or methyl;
Z is a unit chosen from:
i) (biscyclohexyl)methylene having the formula: 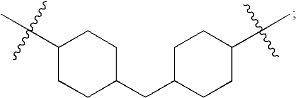
ii) 4-phenylene having the formula: 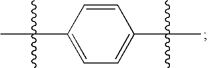
iii) 4,4′-biphenyl having the formula: 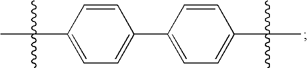
or
iv) an alkylene unit having the formula: 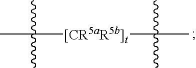
wherein each R^{5a} and R^{5b} is independently chosen from hydrogen, methyl, ethyl, or —CO₂R⁶, wherein R⁶ is chosen from hydrogen, methyl, or ethyl; the index t is from 2 to 12.

4. A medical device according to claim 3 wherein the biodegradable polymer has the formula: 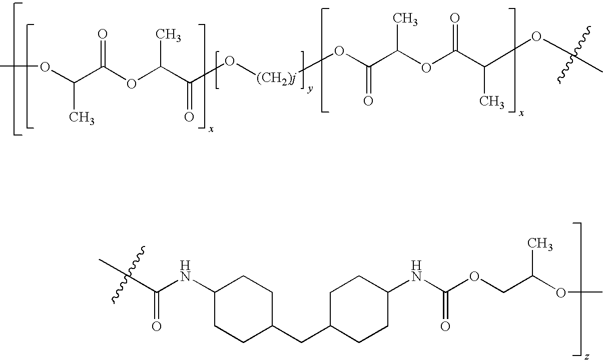
wherein j is from 2 to 6;
the ratio of the index x to y is from 1.5:1 to 2.5:1; the index z is from 2 to 5.

5. A medical device according to claim 3 wherein the biodegradable polymer has the formula: 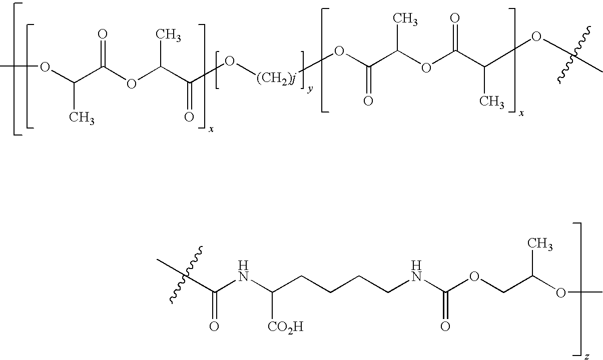
wherein j is from 2 to 6;
the ratio of the index x to y is from 1.5:1 to 2.5:1; the index z is from 2 to 5.

6. A medical device according to claim 1 wherein the biodegradable polymer is a polyurethane having the formula: 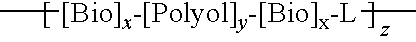
wherein:
Bio is a residue having the formula; 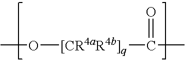
each R^{4a} and R^{4b} is a unit independently chosen from hydrogen, methyl, or ethyl;
Polyol is a residue having the formula: [symbol]
O—[(CR^{1a}R^{1b})ₙO]ₘ—[CR^{2a}R^{2b}]ⱼ [symbol]
each R^{1a}, R^{1b}, R^{2a}, and R^{2b} is independently chosen from hydrogen, methyl, or ethyl; the index j is from 1 to 10; the index m is from 0 to 100; the index n is from 2 to 6; and
L is a linking residue having the formula: 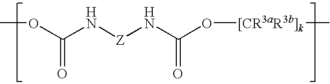
each R^{3a} and R^{3b} is independently chosen from hydrogen, methyl, or ethyl;
Z is a unit comprising one or more:
i) substituted or unsubstituted C₁-C₁₂ linear or branched hydrocarbyl:
i) substituted or unsubstituted C₃-C₇ carbocyclic rings;
ii) substituted or unsubstituted C₁-C₉ heteroaryl rings;
iii) substituted or unsubstituted C₁-C₉ heterocyclic rings; or
v) substituted or unsubstituted C₆, C₁₀, or C₁₋₄ aryl rings;
the index k is from 2 to 10;
the index x is from 1 to 12; the index y is from 1 to 10; the index z is from 2 to 50.

7. A medical device according to claim 1 wherein the biodegradable polymer is a polyurethane having the formula: 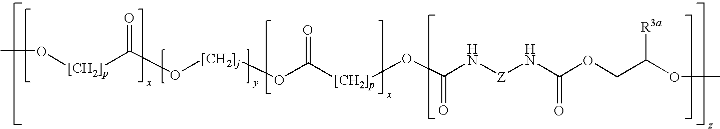
wherein R^{3a} is hydrogen or methyl;
Z is a unit chosen from:
i) (biscyclohexyl)methylene having the formula: 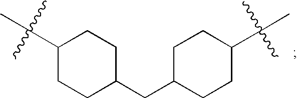
ii) 4-phenylene having the formula: 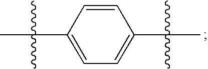
iii) 4,4′-biphenyl having the formula: 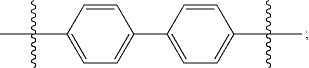
iv) an alkylene unit having the formula: 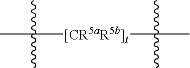
wherein each R^{5a} and R^{5b} is independently chosen from hydrogen, methyl, ethyl, or —CO₂R⁶, wherein R⁶ is chosen from hydrogen, methyl, or ethyl; the index t is from 2 to 12.

8. A medical device according to claim 7 wherein the biodegradable polymer has the formula: 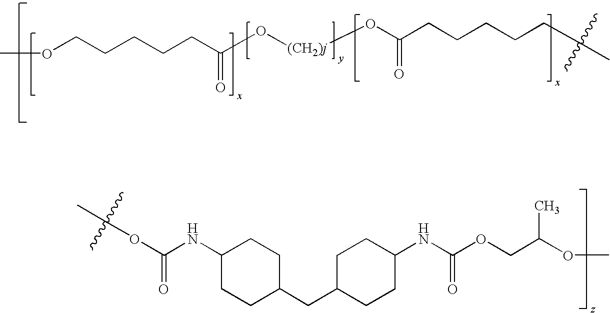
wherein j is from 2 to 6;
the ratio of the index x to y is from 1.5:1 to 2.5:1; the index z is from 2 to 5.

9. A medical device according to claim 7 wherein the biodegradable polymer has the formula: 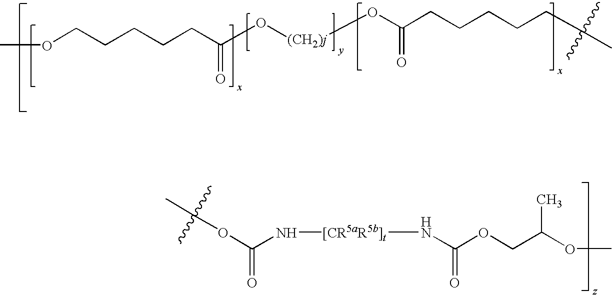
wherein at least one R^{5a} is —CO₂H; j is from 2 to 6; t is 4 or 5; the ratio of the index x to y is from 1.5:1 to 2.5:1; the index z is from 2 to 5.

10. A medical device according to claim 1 wherein the biodegradable polymer is formed from the reaction of one or more monomers chosen from:
i) a cyclic ester having the formula: 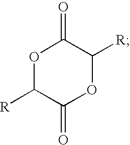
each R is a unit independently chosen from hydrogen, methyl, or ethyl; of
ii) a lactol having the formula: 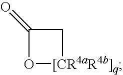
each R^{4a} and R^{4b} is a unit independently chosen from hydrogen, methyl, ethyl; the index q is from 1 to 7;
iii) a polyol having the formula:
HO[(CR^{1a}R^{1b})ₙO]ₘ[(CR^{2a}R^{2b}]ⱼOH
each R^{1a}, R^{1b}, R^{2a}, and R^{2b} is independently chosen from hydrogen, methyl, or ethyl; the index j is from 1 to 10; the index m is from 0 to 100; the index n is from 2 to 6;
iv) a diisocyanate having the formula:
OCN—Z—NCO
Z is a unit comprising one or more:
i) substituted or unsubstituted C₁-C₁₂ linear or branched hydrocarbyl:
ii) substituted or unsubstituted C₃-C₇ carbocyclic rings;
iii) substituted or unsubstituted C₁-C₉ heteroaryl rings;
iv) substituted or unsubstituted C₁-C₉ heterocyclic rings; or
v) substituted or unsubstituted C₆, C₁₀, or C₁₋₄ aryl rings; and
v) a diol having the formula:
HO(CR^{3a}R^{3b})ₖOH
each R^{3a} and R^{3b} is independently chosen from hydrogen, methyl, or ethyl; the index k is from 2 to 10.

11. A medical device according to claim 1 wherein the therapeutic agent is effective for preventing or treating one or more of the following:
a) a sexually transmitted disease;
b) pregnancy;
c) endometriosis;
d) vaginal infections;
e) polycystic ovarian symptoms;
f) diabetes;
g) overactive bladder; or
h) vaginal atrophy.

12. A medical device according to claim 8 wherein the therapeutic agent is effective against HIV.

12a. A medical device according to claim 11 wherein there are a plurality of therapeutic agents.

13. A medical device according to claim 9 wherein the therapeutic agent is
a. a non-nucleoside reverse transcriptase inhibitor
b. a nucleoside reverse transcriptase inhibitor

14. A medical device according to claim 10 wherein the non-nucleoside reverse transcriptase inhibitor is
a. TMC 120.
b. Tenofovir

15. A medical device according to claim 1 in the shape of a ring.

16. A medical device according to claim 1 in the shape of a ring having a first segment and a second segment, each segment comprising about one-half of the circumference of the ring, wherein
i) the therapeutic agent is dispersed within the first segment and the second segment of the ring; and
ii) the ring is capable of releasing into a person using the medical device an effective amount of the therapeutic agent for at least about 30-90 days.

17. A medical device according to claim 1 in the shape of a ring having a first segment and a second segment, each segment comprising about one-half of the circumference of the ring; the medical device further comprising a second therapeutic agent, wherein
i) the therapeutic agent is dispersed within the first segment of the ring;
ii) the second therapeutic agent is dispersed within the second segment of the ring; and
ii) the ring is capable of releasing into a person using the medical device an effective amount of the therapeutic agent and the second therapeutic agent for at least about 30 days.

18. A medical device according to claim 13 wherein the first segment and the second segment of the ring form a continuous ring.

19. A medical device according to claim 1 formed by two concentric rings, an outer ring and an inner ring, the two rings in register which each other; wherein the outer ring comprises at least an effective amount of the therapeutic agent.

20. A medical device according to claim 16 further comprising a second therapeutic agent.

21. A medical device according to claim 17 wherein the second therapeutic agent is disperse within the inner concentric ring.

22. A medical device according to claim 1 wherein a first biodegradable polymer layer comprising a therapeutic agent enrobes a second biodegradable polymer core comprising no therapeutic agent.

23. A medical device according to claim 1 wherein a first biodegradable polymer layer which does not comprise a therapeutic agent enrobes a second biodegradable polymer core comprising a therapeutic agent.

24. A medical device according to claim 1 wherein a first biodegradable polymer layer comprising a therapeutic agent enrobes a second biodegradable polymer core comprising no therapeutic agent wherein the first polymer layer has a higher melting point than the polymer core.

25. A medical device according to claim 1 wherein a first biodegradable polymer layer which does not comprise a therapeutic agent enrobes a second biodegradable polymer core comprising a therapeutic agent wherein the first polymer layer has a higher melting point than the polymer core.

26. A method for treating or preventing a sexually transmitted disease comprising providing to a patient in need of treatment or in need of prevention of a sexually transmitted disease a medical device according to claim 1.

27. A method for treating or preventing a sexually transmitted disease comprising providing to a patient in need of treatment or in need of prevention of a sexually transmitted disease a medical device according to claim 16.

28. A method according to claim 27 wherein the therapeutic agent is effective against HIV.

29. A medical device according to claim 28 wherein the therapeutic agent is a non-nucleoside reverse transcriptase inhibitor.

30. A medical device according to claim 29 wherein the non-nucleoside reverse transcriptase inhibitor is TMC 120.

31. A method for treating or preventing a sexually transmitted disease comprising providing to a patient in need of treatment or in need of prevention of a sexually transmitted disease a medical device according to claim 17.

32. A method according to claim 31 wherein the therapeutic agent is effective against HIV.

33. A medical device according to claim 32 wherein the therapeutic agent is a non-nucleoside reverse transcriptase inhibitor.

34. A medical device according to claim 33 wherein the non-nucleoside reverse transcriptase inhibitor is TMC 120.

35. A method for treating or preventing a sexually transmitted disease comprising providing to a patient in need of treatment or in need of prevention of a sexually transmitted disease a medical device according to claim 19.

36. A method according to claim 35 wherein the therapeutic agent is effective against HIV.

37. A medical device according to claim 36 wherein the therapeutic agent is a non-nucleoside reverse transcriptase inhibitor.

38. A medical device according to claim 37 wherein the non-nucleoside reverse transcriptase inhibitor is TMC 120.

39. A method for treating or preventing a sexually transmitted disease comprising providing to a patient in need of treatment or in need of prevention of a sexually transmitted disease a medical device according to claim 23.

40. A method according to claim 39 wherein the therapeutic agent is effective against HIV.

41. A medical device according to claim 40 wherein the therapeutic agent is a non-nucleoside reverse transcriptase inhibitor.

42. A medical device according to claim 41 wherein the non-nucleoside reverse transcriptase inhibitor is TMC 120.

43. A method for treating or preventing a sexually transmitted disease comprising providing to a patient in need of treatment or in need of prevention of a sexually transmitted disease a medical device according to claim 24.

44. A method according to claim 43 wherein the therapeutic agent is effective against HIV.

45. A medical device according to claim 44 wherein the therapeutic agent is a non-nucleoside reverse transcriptase inhibitor.

46. A medical device according to claim 45 wherein the non-nucleoside reverse transcriptase inhibitor is TMC 120.
